(19) United States
(12) Patent Application Publication
(10) Pub. No.: US 2026/0069143 A1
(43) Pub. Date: Mar. 12, 2026

(54) TRANSITION EDGE BASED EYE REGISTRATION

(71) Applicant: Alcon Inc., Fribourg (CH)

(72) Inventor: Martin Gruendig, Rangsdorf (DE)

(21) Appl. No.: 19/322,256

(22) Filed: Sep. 8, 2025

Related U.S. Application Data
(60) Provisional application No. 63/692,825, filed on Sep. 10, 2024.

(51) Int. Cl. A61B 3/14; A61B 3/10; A61B 3/11; G06T 5/20; G06T 7/13

(52) U.S. Cl. CPC A61B 3/14; A61B 3/1005; A61B 3/112; G06T 5/20; G06T 7/13; G06T 2207/20132

(57) ABSTRACT

The present disclosure relates to operations that include identifying a first transition-edge portion of a first image of an eye that corresponds to a transition edge of the eye. The operations may also include determining, based on first-portion pixel data corresponding to the first transition-edge portion, a first transition-edge representation of the transition edge. The operations may include identifying a second transition-edge portion of a second image of the eye that corresponds to the transition edge. In addition, the operations may include determining, based on second-portion pixel data corresponding to the second transition-edge portion, a second transition-edge representation of the transition edge. Moreover, the operations may include determining an alignment registration between the eye as depicted in the first image and the second image based on a transition-edge based registration that is based on a comparison between the first transition-edge representation and the second transition-edge representation.

### CROSS-REFERENCE TO RELATED APPLICATION

This application claims priority to U.S. Provisional Patent Application No. 63/692,825, filed on Sep. 10, 2024, the disclosure of which is incorporated herein by reference in its entirety.

### BACKGROUND

Images of eyes are often used in ophthalmology to diagnose certain eye conditions. Such images may also be used to determine a course of action for treating such conditions. Often ophthalmic surgical systems may be used as part of carrying out a specified treatment plan in which such systems may use the diagnostic images in carrying out such plans. However, the subject eye may be oriented differently during the course of treatment than during the diagnostic stages. As such, the orientation of the subject eye as depicted in the diagnostic image and as used by the ophthalmic surgical system may differ from the orientation of the subject eye during treatment.

### SUMMARY

The present disclosure relates to operations that include identifying a first transition-edge portion of a first image of an eye that corresponds to a transition edge of the eye. The operations may also include determining, based on first-portion pixel data corresponding to the first transition-edge portion, a first transition-edge representation of the transition edge. The operations may include identifying a second transition-edge portion of a second image of the eye that corresponds to the transition edge. In addition, the operations may include determining, based on second-portion pixel data corresponding to the second transition-edge portion, a second transition-edge representation of the transition edge. Moreover, the operations may include determining an alignment registration between the eye as depicted in the first image and the second image based on a transition-edge based registration that is based on a comparison between the first transition-edge representation and the second transition-edge representation.

### BRIEF DESCRIPTION OF THE DRAWINGS

The present disclosure relates to systems and methods for performing registration of an eye based on transition edges within the eye, wherein:

FIG. 1 illustrates an example system configured to perform transition-edge based eye registration, in accordance with one or more embodiments of the present disclosure;

FIG. 2 is a flow diagram illustrating a method for determining an eye registration, in accordance with one or more embodiments of the present disclosure;

FIG. 3A is a flow diagram illustrating a process for identifying a transition-edge portion corresponding to a transition edge of an eye, in accordance with one or more embodiments of the present disclosure;

FIG. 3B illustrates an example image of an eye, in accordance with one or more embodiments of the present disclosure;

FIG. 3C illustrates a pupil-edge portion of an image, in accordance with one or more embodiments of the present disclosure;

FIG. 4A is a flow diagram illustrating an example process for determining a transition-edge representation, in accordance with one or more embodiments of the present disclosure;

FIG. 4B illustrates an example graph of a pupil-edge representation, in accordance with one or more embodiments of the present disclosure;

FIG. 5A is a flow diagram illustrating an example process for determining an alignment registration, in accordance with one or more embodiments of the present disclosure; and

FIG. 5B illustrates example graphs of pupil-edge representations, in accordance with one or more embodiments of the present disclosure;

FIG. 5C illustrates an example graph of a correlation representation that may correspond to a correlation distribution, in accordance with one or more embodiments of the present disclosure;

FIG. 6 is a block diagram of an example ophthalmic surgical system, in accordance with one or more embodiments of the present disclosure; and

FIG. 7 is a block diagram of an example computing system suitable for use in implementing one or more embodiments of the present disclosure.

### DETAILED DESCRIPTION

One or more embodiments of the present disclosure relate to registration of an eye. In general, reference to “eye registration” in the present disclosure may refer to determining a particular orientation of the eye with respect to a particular reference orientation. In some embodiments, the reference orientation may correspond to the orientation of the eye in a first image of the eye. In these and other embodiments, the registration of the eye may include determining an alignment registration between the first image and a second image of the eye.

According to one or more embodiments of the present disclosure, the eye registration may be used and/or performed by one or more ophthalmic surgical systems configured to perform one or more treatment tasks with respect to eyes. For example, in some instances a diagnostic image may be taken of an eye. The diagnostic image may be used to diagnose one or more conditions of the eye and/or to determine a treatment plan for the eye. Additionally or alternatively, the eye may be in a particular state during the capture of the eye for the diagnostic image. For example, in some instances the pupil of the eye may be relatively constricted and/or oriented in a particular manner. In these and other embodiments, the orientation of the eye may be based on how the patient's head is turned and/or whether the patient's head is upright or laying down. For example, eyes may rotate within the eye socket-often referred to as cyclotorsion-due to rotations, tilts, positioning, etc. of the head.

In some instances, the state of the eye during treatment may be different than the state of the eye in the diagnostic image. For example, the pupil may be significantly more dilated (e.g., through dilating eye drops) or less dilated (e.g., due to brighter illumination during treatment as compared to diagnosis) than during diagnosis. As another example, the pupil may be oriented differently—e.g., the patient's head may have been upright during diagnosis and may be laying down during treatment.

Additionally or alternatively, the ophthalmic surgical system may use the diagnostic image as a reference for performing one or more treatment tasks with respect to the eye. For example, the diagnostic image may identify portions of the eye for which certain treatment procedures may be performed. As such, one or more embodiments of the present disclosure may relate to the ophthalmic surgical system performing eye registration with respect to the diagnostic image (e.g., in which the orientation of the eye in the diagnostic image is used as the reference orientation) such that the ophthalmic surgical system identifies which portions of the eye as currently viewed by the ophthalmic surgical system correspond to the portions for which the procedures are to be performed. In these and other embodiments, the eye registration may include a cyclotorsion registration that may be used to determine or indicate an amount of cyclotorsion during treatment as compared to in the diagnostic image. Doing so may facilitate alignment of a surgical device at the time of treatment for a treatment task which was planned based on the diagnostic image.

In particular, as described in further detail, systems and methods may relate to performing eye registration based on transition edges of the eye. In the present disclosure, reference to a “transition edge” may refer to edges or boundaries between discreet portions of the eye. For instance, an example transition edge may include the outer edge of the pupil that borders the inner portion of the iris-referred to in the present disclosure as the “pupil edge.” Another example transition edge may include the inner edge of the sclera that also borders the outer portion of the iris-referred to in the present disclosure as the “scleral edge.”

Using transition edges for eye registration may help improve the accuracy of the eye registration. For example, many common techniques of eye registration are based mainly on matching features related to the iris. However, such feature matching may be difficult in some instances. For example, in many instances the eye for which a procedure may be performed (also referred to as a “subject eye” in the present disclosure) may be in a relatively undilated state when a corresponding diagnostic image is captured. Further, the pupil of the subject eye may be artificially dilated (e.g., via dilation eyedrops) during the procedure, which may distort many features of the iris and accordingly render iris-based registration difficult or unusable. As a further example, for some treatments the pupil of the subject eye may be more dilated when the corresponding diagnostic image is captured (e.g., due to dark lighting conditions or artificial dilation) while the eye may be in an undilated state (e.g., due to bright illumination) for the treatment.

Additionally or alternatively, different illumination or lighting setups that are used with respect to diagnostic images as compared to during treatment may cause different features of the iris to appear differently, which may also cause issues with iris-based registration. However, the features of the transition edges may not undergo the same degree of distortion due to changes in pupil dilation and/or lighting and illumination setups such that transition-edge based registration may help improve overall registration accuracy in instances in which iris-based registration may be deficient.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to performance of eye registration in the context of ophthalmic surgical systems. However, such uses are not meant to be limiting such that the eye registration described may be used in any number of different contexts and applications where it may be helpful or applicable.

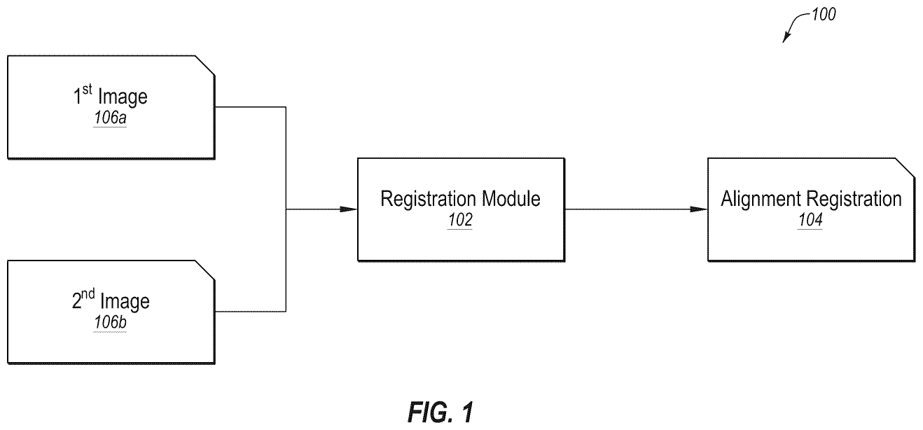

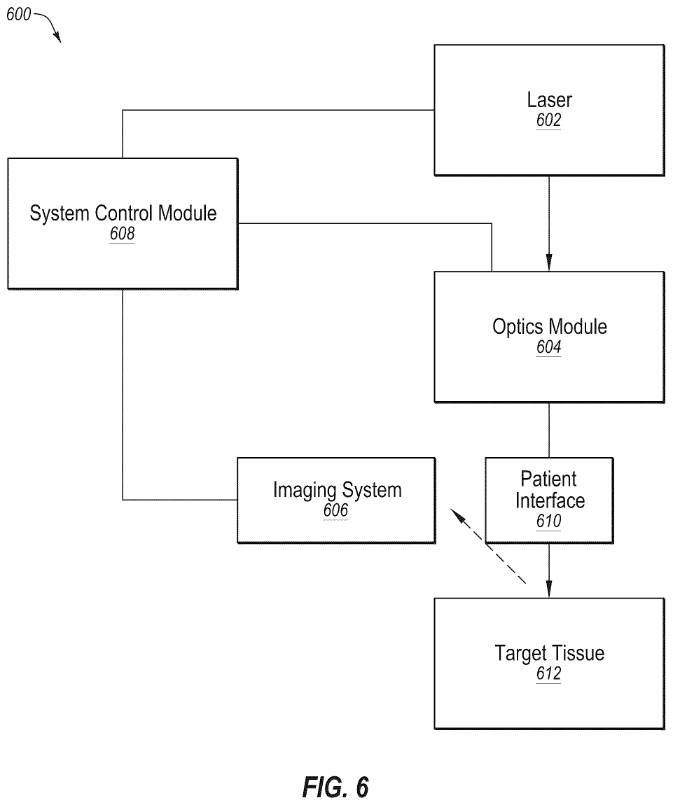

FIG. 1 illustrates an example system 100 configured to perform transition-edge based eye registration, in accordance with one or more embodiments of the present disclosure. In some embodiments, the system 100 may be deployed with respect to an ophthalmic surgical system, such as that described in further detail with respect to FIG. 6 of the present disclosure. The system 100 may include a registration module 102 configured to determine an alignment registration 104 between a first image 106a and a second image 106b.

The first image 106a may be obtained using a first camera which may include any suitable image sensor configured to detect multiple different wavelengths of light—visible and/or non-visible (e.g., infrared)—received through a lens of the first camera. Further, the first camera may be configured to generate the first image 106a based on the detected light in any suitable manner. For example, the first image 106a may include an infrared image of the eye, a visible image of the eye or any suitable combination thereof. In the present disclosure reference to a camera “capturing” an image may include the camera detecting light and generating a corresponding image based on the detected light. Further, in the context of describing the eye as depicted in images, reference to “the eye” or different portions of the eye, such as the pupil, iris, sclera, etc. may refer to the portions of the images that depict the eye, the pupil, the iris, the sclera, etc. and may not necessarily be referring to the eye itself.

Additionally or alternatively, the first image 106a may correspond to a first state of the eye. In some embodiments, the first state may correspond to a first dilated state of the pupil of the eye (also referred to as a dilated state of the eye). For example, in some instances, the first state may include the eye being in a relatively constricted state or in a dilated state (e.g., artificially dilated via dilation eyedrops). In the present disclosure, reference to an eye being “constricted” as compared to being “dilated” may refer to instances in which a difference in pupil dilation satisfies a certain threshold. For example, instances in which the size of the pupil enlarges from a first size to a second size by a threshold percentage (e.g., 50% or more) may be such that a first state corresponding to the first size may be considered “constricted” and a second state corresponding to the second size may be considered “dilated.” For example, a pupil that is referred to as being in a “dilated” state may not actually be fully dilated but may merely be dilated more than in a different state that is referred to as being “constricted.”

Additionally or alternatively, the first state of the eye may include an orientation and/or position of the eye in the first image 106a. In these and other embodiments, the first state of the eye may include an orientation of the eye in the eye socket when a corresponding head is in an upright or laying down position.

In these and other embodiments, the first state of the eye may include a first lighting condition associated with the eye. For example, the first lighting condition may include an ambient light condition of the light around the eye. Additionally or alternatively, the first lighting condition may include an illumination condition with respect to a light that is specifically directed toward the eye. The light that is directed toward the eye may include visible and/or non-visible (e.g., infrared) light. For example, in some embodiments, the eye may be illuminated with an infrared illumination as part of capturing of the first image 106a. Additionally or alternatively, the eye may be illuminated with white light.

Further, the first image 106a may include first image data associated therewith. The first image data may include information about the first image 106a. For example, the first image 106a may be divided into pixels that are used to create or represent the first image 106a. The pixels may include data associated therewith (referred to as “pixel data”) that describe the features of light that are to be depicted by the pixels. For example, the pixel data may include wavelength information related to the wavelength of detected light corresponding to respective pixels. The wavelength information may indicate a hue or color of visible detected light that corresponds to the respective pixels. Additionally or alternatively, the pixel data may include intensity data that may indicate an intensity (e.g., brightness, magnitude of waveform, etc.) of the detected light corresponding to the respective pixels. In some embodiments the intensity data may specifically correspond to infrared intensity, which may correspond to an infrared illumination in some instances. Additionally or alternatively, the intensity data may correspond to a white light intensity, which may correspond to a white light illumination in some instances. In these and other embodiments, the white light may be broken into different color channels—such as a red channel, a green channel, and a blue channel—and the intensity data may indicate intensities of one or more of the individual channels. Note that in the present disclosure, reference to an image may also refer to corresponding image data associated therewith.

In some embodiments the first image 106a may be a diagnostic image of the eye. The first image 106a may accordingly be used to diagnose one or more conditions (also referred to in the present disclosure as ophthalmic conditions) and/or determine a treatment plan for treatment of the conditions. Additionally or alternatively, the first camera used to capture the first image 106a may be included with any applicable machine or system that may be used for diagnosis of ophthalmic conditions.

The second image 106b may be obtained using a second camera which may include any suitable image sensor configured to detect multiple different wavelengths of light—visible and/or non-visible (e.g., infrared)—received through a lens of the second camera. Further, the second camera may be configured to generate the second image 106b based on the detected light in any suitable manner. In some embodiments, the second camera may be the same camera as the first camera used to capture the first image 106a. Additionally or alternatively, the second camera may be a different camera. In some embodiments, the second image 106b and/or the first image 106a may be one image in a video.

In some embodiments, the second image 106b may correspond to a second state of the eye. In some embodiments, the second state may correspond to a second dilated state of the pupil of the eye. For example, similar to the first state, in some instances, the second state may include the eye being in a relatively constricted state or in a dilated state. Additionally or alternatively, the second state of the eye may include an orientation and/or position of the eye in the second image 106b. In these and other embodiments, the second state of the eye may include an orientation of the eye in the eye socket when a corresponding head is in an upright or laying down position. In these and other embodiments, the second state of the eye may include a second lighting condition associated with the eye that may be selected from options that are analogous to those used for the first lighting condition. Further, the second image 106b may include second image data associated therewith. The second image data may include similar or analogous content to the first image data.

In some embodiments the second image 106b may be associated with performance of a treatment procedure with respect to the eye. For example, in some embodiments, the second camera used to capture the second image 106b may be included in an ophthalmic surgical system configured to perform one or more ophthalmic treatment tasks with respect to the eye. For instance, the ophthalmic surgical system may include a laser configured to perform one or more tasks with respect to the eye, such as a femtosecond laser assist cataract surgery (FLACS) device, a laser assisted in situ keratomileusis (LASIK) surgical device, a small incision lenticule extraction (SMILE) surgical device, or any other laser-based surgical device. The ophthalmic surgical system may use the second image 106b to detect the eye, a location of the eye, etc. Additionally or alternatively, as discussed in further detail in the present disclosure, the second image 106b may also be used to identify the locations of the portions of the eye on which the treatment tasks may be performed.

The registration module 102 may be configured to process the first image 106a and the second image 106b to determine the alignment registration 104. In some embodiments, the registration module 102 may be included with the ophthalmic surgical system that may be used to capture the second image 106b.

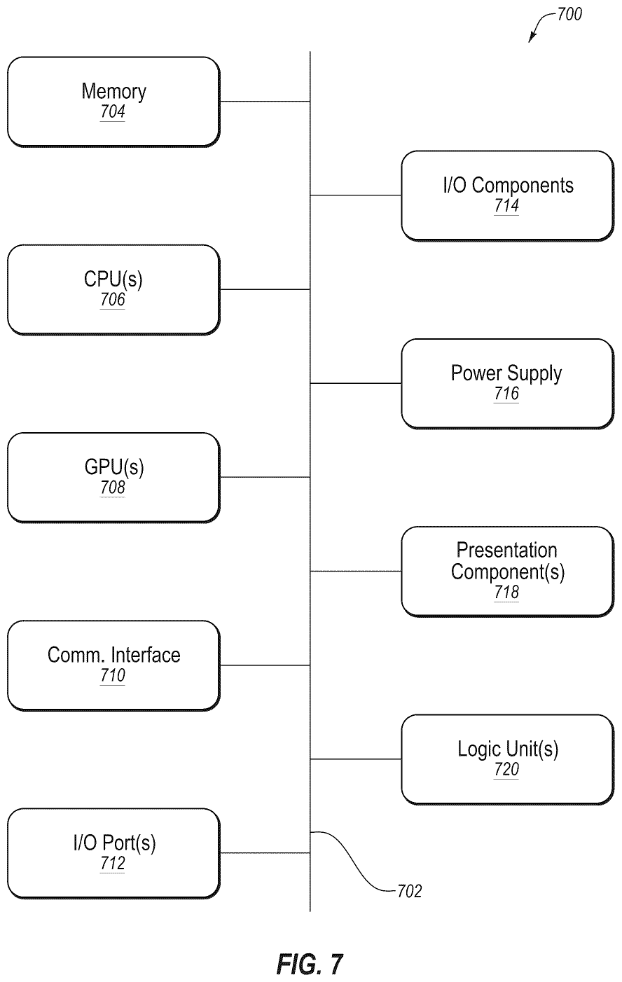

In some embodiments, the registration module 102 may include code and routines configured to allow a computing system to perform one or more registration operations. Additionally or alternatively, the registration module 102 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the registration module 102 may be implemented using a combination of hardware and software. In the present disclosure, operations described as being performed by the registration module 102 may include operations that the registration module 102 may direct a corresponding computing system to perform. In these or other embodiments, the registration module 102 may be implemented by one or more computing systems, such as that described in further detail with respect to FIG. 7 of the present disclosure.

In some embodiments, the alignment registration 104 may include a registration between the eye as depicted in the first image 106a and the eye as depicted in the second image 106b. For example, in some embodiments, the alignment registration may include one or more alignment parameters that may indicate the relative orientation and/or position of the eye between the first image 106a and the second image 106b. In these and other embodiments, the alignment parameters may be used to align the eye in the first image 106a with the eye in the second image 106b. For example, the alignment parameters may include one or more rotations, translations, scaling adjustments, etc. applied to one or more of the first image 106a or the second image 106b and that may be used to identify which portions of the first image and the second image depict the same portions of the eye. In these and other embodiments, the alignment registration 104 may accordingly be used to indicate where certain portions of the eye indicated for treatment (also referred to as “treatment portions of the eye”) in the first image 106a may be located in the second image 106b. In the present disclosure, reference of alignment of images may refer to the alignment of the eye as depicted in the images.

Additionally or alternatively, in some embodiments, the alignment registration 104 may include one or more alignment parameters that may be used to determine a registration with respect to the eye itself, such that the alignment registration 104 may include and/or be referred to as an eye registration. For example, in some embodiments, the second image 106b may be captured in real time during performance of an ophthalmic surgical procedure by the ophthalmic surgical system. Further, based on a known location of the second camera, a location of the eye in the second image 106b, and a known relative location of the second camera with respect to a laser beam used to perform treatment tasks, the relative location of the eye itself with respect to the laser beam may be determined. In addition, as indicated above, the alignment registration 104 may be used to identify the locations of certain portions of the eye in the second image 106b (e.g., treatment portions of the eye), which may then be used to determine the relative locations of the portions with respect to the laser beam.

In these and other embodiments, the alignment registration 104 may include a cyclotorsion registration with respect to the eye. For example, the first state of the eye with respect to the first image 106a may be based on the head of the patient being upright during capture of the first image 106a. Further, the second state of the eye with respect to the second image 106b may be based on the head of the patient laying down during capture of the second image 106b. As such, the eye may be rotated to some degree in the second image 106b as compared to the first image 106a. The alignment registration 104 may indicate the degree of such cyclotorsion between the first image 106a and the second image 106b as a cyclotorsion registration. In some embodiments, the cyclotorsion registration may be indicated by a radial measurement (e.g., degrees, radians) that may indicate the amount of rotation.

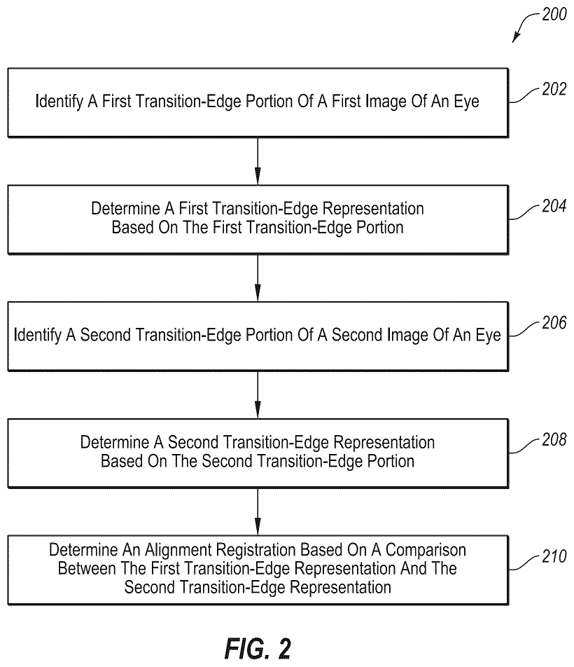

In some embodiments and as indicated above, the registration module 102 may be configured to determine the alignment registration 104 based on one or more transition edges of the eye. For example, in some embodiments, the alignment registration may be based on an outer edge of the pupil that borders the iris and/or an inner edge of the sclera that borders the iris. Additionally or alternatively, in some embodiments, the registration module 102 may be configured to determine the alignment registration 104 based on transition edges as discussed with respect to one or more operations described with respect to FIGS. 2-5B.

In some embodiments, the registration module 102 may be configured to determine the alignment registration 104 based primarily on one or more transition edges. Additionally or alternatively, the registration module 102 may be configured to determine the alignment registration 104 based on both transition edges and using one or more other registration techniques.

For example, in some embodiments, the registration module 102 may be configured to determine the alignment registration 104 based on any applicable iris registration technique in addition to being based on transition edges. For instance, in some embodiments, the registration module 102 may be configured to identify within the first image 106a and the second image 106b certain portions of the iris that may have unique characteristics and may determine the alignment registration 104 at least partially based on the relative locations of one or more of the same unique characteristics in both the first image 106a and the second image 106b.

Modifications, additions, or omissions may be made to FIG. 1 without departing from the scope of the present disclosure. For example, the system 100 may include more or fewer elements depending on the implementation. For instance, the registration module 102 may be configured to determine multiple different alignment registrations 104 based on multiple comparisons between the first image 106a and multiple other second images. Additionally or alternatively, although described as being deployed with respect to an ophthalmic surgical system, in some embodiments, the registration module 102 may be deployed with respect to any suitable application where registration of an eye may be applicable.

FIG. 2 is a flow diagram illustrating a method 200 for determining an alignment registration, in accordance with one or more embodiments of the present disclosure. One or more operations of the method 200 may be performed by any suitable system, apparatus, or device such as, for example, the system 100 of FIG. 1, an ophthalmic surgical system such as that described with respect to FIG. 6 of the present disclosure, and/or a computing system such as that described with respect to FIG. 7 of the present disclosure.

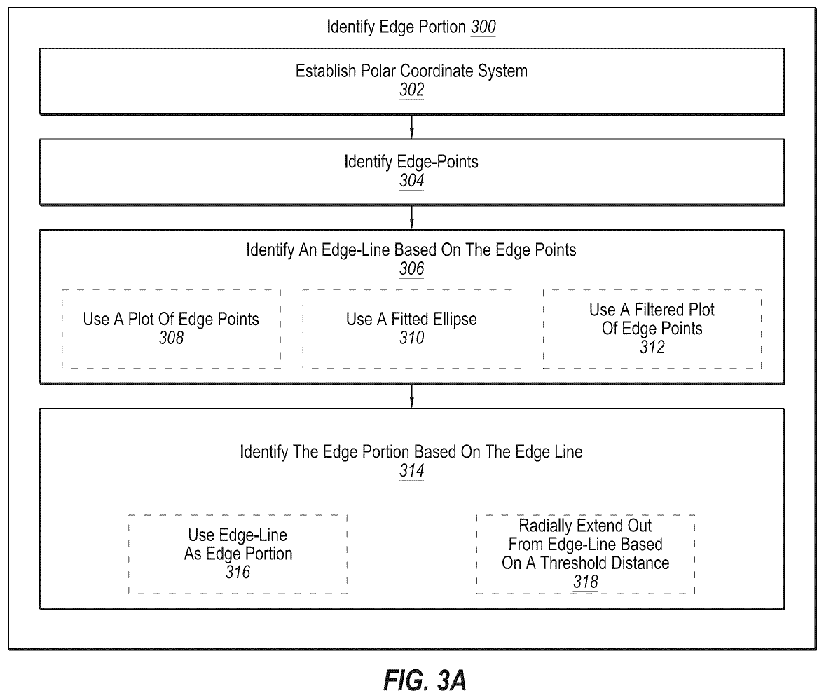

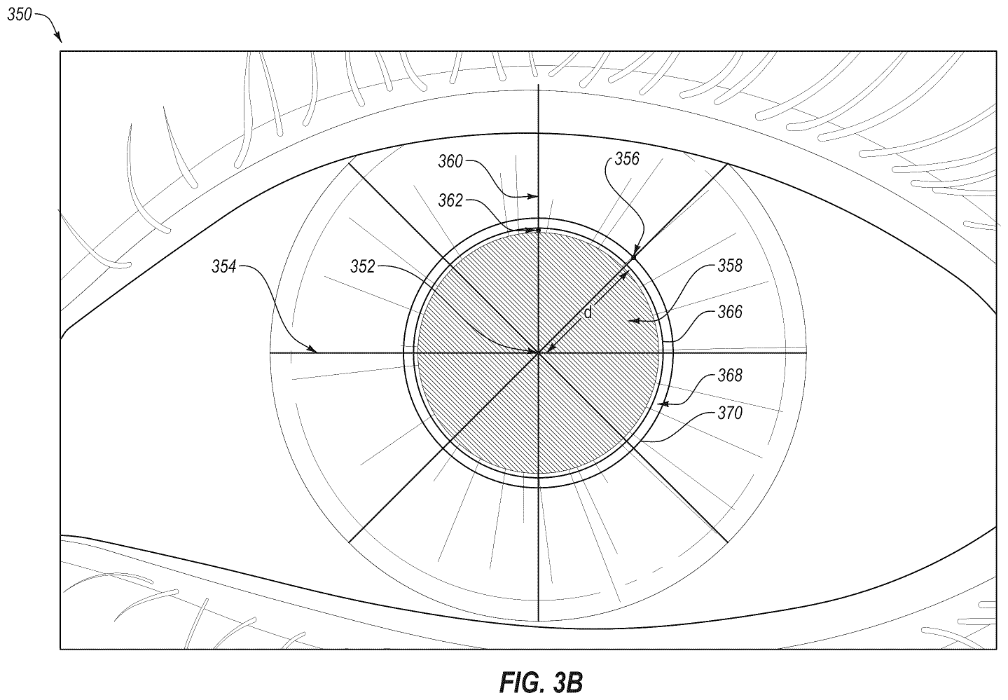

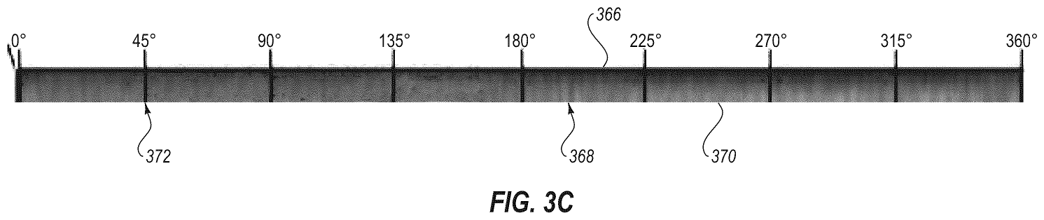

At block 202, a first transition-edge portion of a first image of an eye may be identified. In some embodiments, the first transition-edge portion may correspond to a transition edge of the eye. For example, in some embodiments, the first transition-edge portion may correspond to an outer edge of the pupil of the eye that borders the iris. Additionally or alternatively, in some embodiments, the first transition-edge portion may correspond to an inner edge of the sclera of the eye that borders the iris. In these and other embodiments, the first image 106a of FIG. 1 may be an example of the first image. Further, in some embodiments, the first transition-edge portion may be identified based on one or more operations of a process described with respect to FIGS. 3A-3D of the present disclosure.

In some embodiments, the first transition-edge portion may correspond to a first range of angular location values. In some embodiments, the first range may include the full circumference of the corresponding edge of the eye (e.g., the outer pupil edge or the inner scleral edge). Additionally or alternatively, the first range may correspond to less than the full circumference of the corresponding edge. For example, the first range may correspond to a 90° slice, a 180° slice, or the like of the first transition-edge portion.

In these and other embodiments, the first range may correspond to a certain percentage of the circumference of the corresponding edge. In some embodiments, the percentage may be set by a user and/or based on a heuristic analysis with respect to a relationship between the percentage and registration accuracy. Additionally or alternatively, the percentage may be based on computing resources that may be available for registration in that registration based on a smaller percentage may use fewer computing resources. In these and other embodiments, the percentage may be based on the density of data corresponding to the first transition-edge portion. For example, the first transition-edge portion that may be used, as discussed in further detail below, to represent the outer edge of the pupil, which may be used to determine the alignment registration. In instances in which the data density is greater (e.g., instances with relatively high resolution), the amount of information that may be used to determine the representation may be greater, which may result in not needing to generate a representation that represents the entire circumference. As a further example, portions of the circumference that are blocked by anatomical features (e.g., eyelashes or eyelid blocking portions of the inner scleral edge) may be excluded from the full 360°.

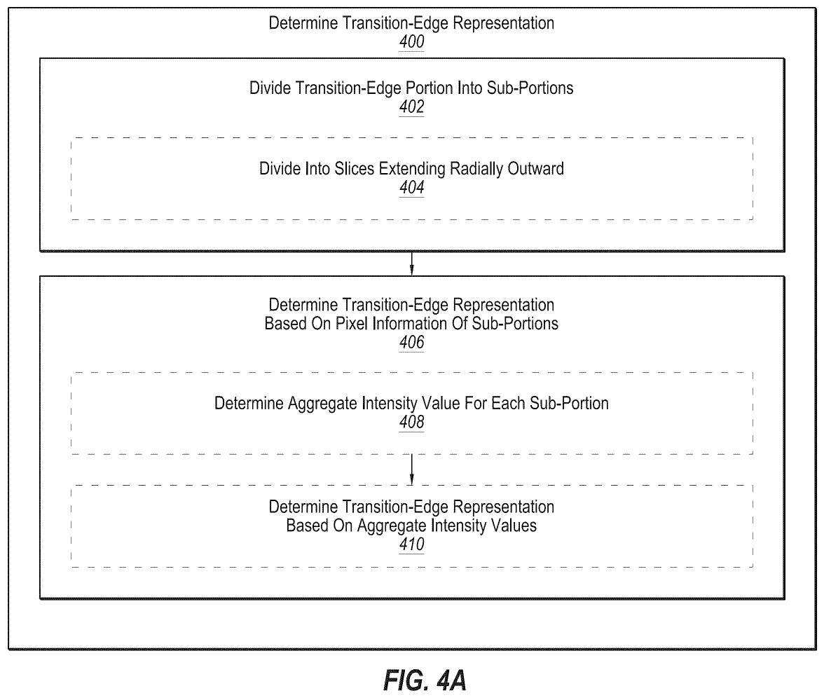

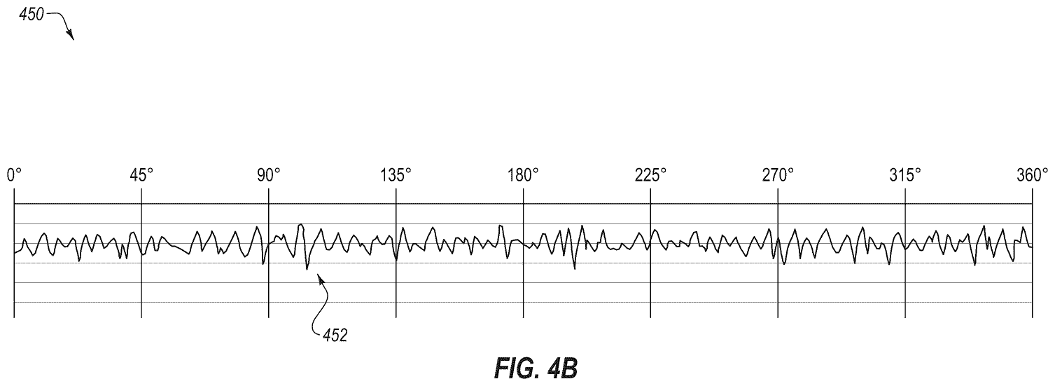

At block 204, a first transition-edge representation may be determined based on the first transition-edge portion. In particular, in some embodiments, first-portion pixel data that corresponds to the first transition-edge portion may be used to generate a representation of the corresponding transition edge. In some embodiments, the first transition-edge representation may be determined based on one or more operations of a process 400 described with respect to FIGS. 4A and 4B of the present disclosure.

In some embodiments, the first transition-edge representation may represent the full circumference of the corresponding transition edge—e.g., in instances in which the first transition-edge portion corresponds to the full circumference of the transition edge. Additionally or alternatively, the first transition-edge representation may represent less than the full circumference of the corresponding transition edge—e.g., in instances in which the first transition-edge portion corresponds to less than the full circumference of the transition edge.

At block 206, a second transition-edge portion of a second image of the eye may be identified. The second transition-edge portion may correspond to the same transition edge as the first transition-edge portion in some embodiments. Additionally or alternatively, in some embodiments, the second image 106b of FIG. 1 may be an example of the second image. In some embodiments, similar to the first transition-edge portion, the second transition-edge portion may be identified based on one or more operations of the process described with respect to FIGS. 3A-3D of the present disclosure.

In some embodiments, the second transition-edge portion may correspond to a second range of angular location values. For example, in some embodiments, the second range may include the full circumference of the transition edge. Additionally or alternatively, the second range may correspond to less than the full circumference of the transition edge. In some embodiments, the second range may be the same as the first range. For example, the amount of the transition edge to which second transition-edge portion may correspond may be similar or the same as that of the first transition-edge portion. Additionally or alternatively, the second range may differ from the first range. 1

At block 208, a second transition-edge representation may be determined based on the second transition-edge portion. In particular, in some embodiments, second-portion pixel data that corresponds to the second transition-edge portion may be used to generate a representation of the transition edge. In some embodiments, the second transition-edge representation, like the first transition-edge representation, may be determined based on one or more operations of the process 400 described with respect to FIGS. 4A and 4B of the present disclosure.

In some embodiments, the second transition-edge representation may represent the full circumference of the transition edge—e.g., in instances in which the second transition-edge portion corresponds to the full circumference of the transition edge. Additionally or alternatively, the second transition-edge representation may represent less than the full circumference of the transition edge—e.g., in instances in which the second transition-edge portion corresponds to less than the full circumference of the transition edge.

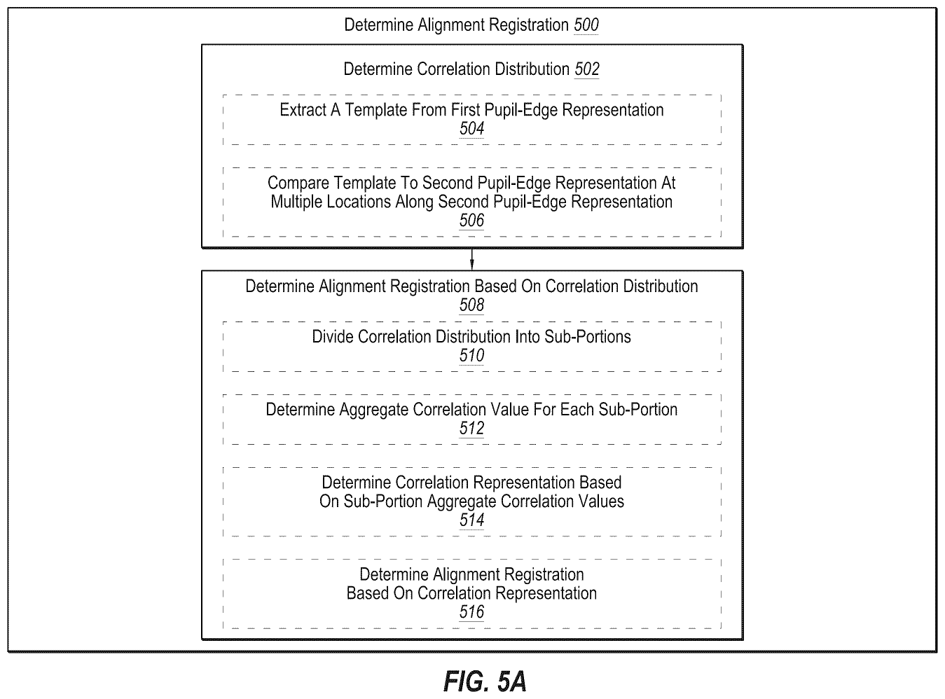

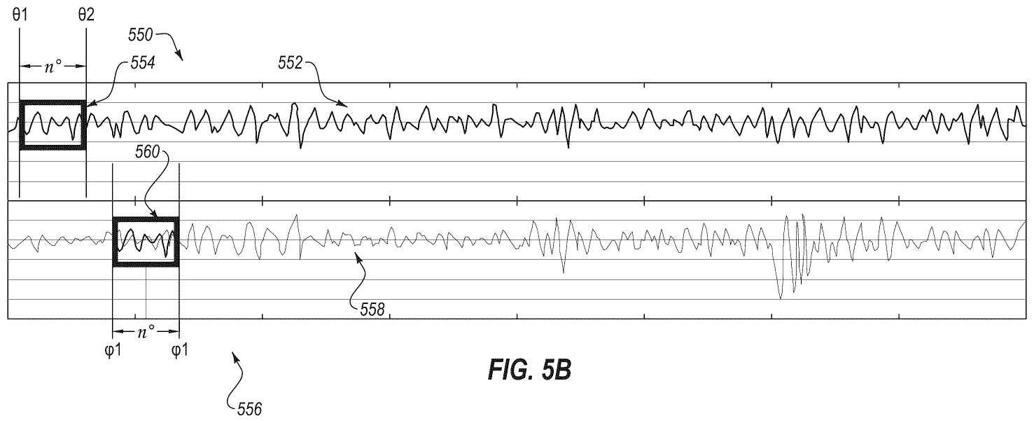

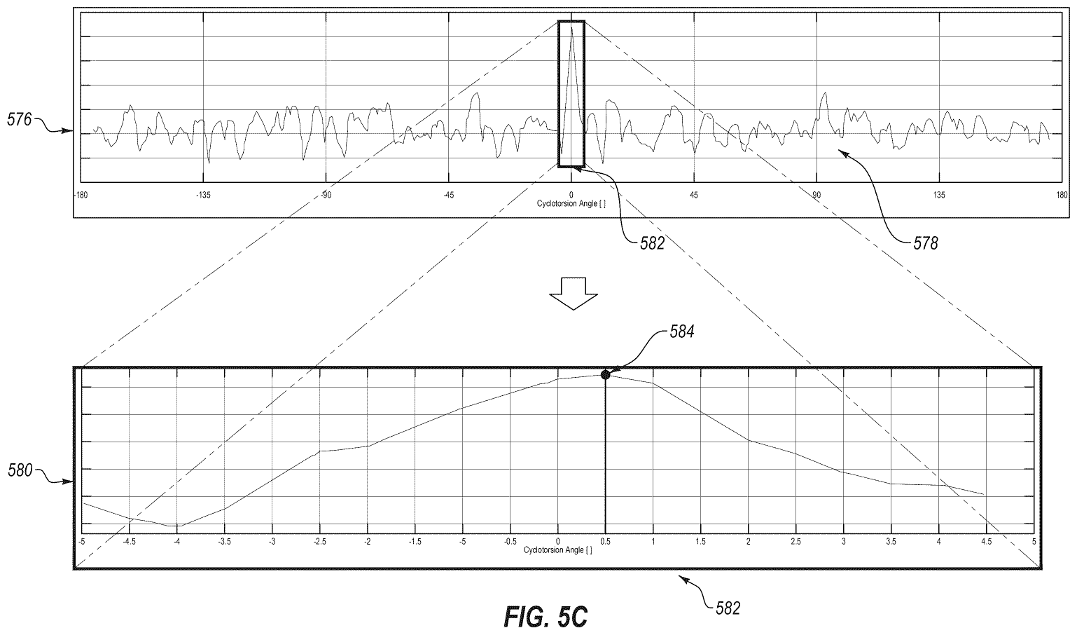

At block 210, an alignment registration may be determined based on a comparison between the first transition-edge representation and the second transition-edge representation. In some embodiments, the alignment registration 104 of FIG. 1 may be an example of the alignment registration. Additionally or alternatively, in some embodiments, the alignment registration may be determined based on one or more operations of a process 500 described with respect to FIGS. 5A and 5B of the present disclosure.

In these and other embodiments, the alignment registration may be based on only a comparison of transition-edge representations corresponding to the outer edge of the pupil (referred to as pupil-edge representations) or only a comparison of transition-edge representations corresponding to the inner edge of the sclera (referred to as scleral-edge representations). Additionally or alternatively, the alignment registration may be based on a first comparison between pupil-edge representations and a second comparison between scleral-edge representations.

In these and other embodiments, the alignment registration may also be determined based on one or more different types of transition edges in addition to any other suitable type of registration such as iris registration. For example, in some embodiments, certain individual points of the eye (e.g., within the iris) may be identified. For instance, one or more points within the iris that satisfy a high intensity value threshold and/or that satisfy a low intensity value threshold may be identified. Additionally or alternatively, a first point in the iris that may have the highest intensity value corresponding thereto (e.g., the brightest point in the iris) may be identified and/or a second point in the iris that may have the lowest intensity value corresponding thereto (e.g., the darkest point in the iris) may be identified. In these and other embodiments, a point-based registration may be performed by matching corresponding points (e.g., the brightest points) in the first image and the second image. In these and other embodiments, the alignment registration may be an aggregate registration that may be based on transition-edge based registration as well as based on point-based registration.

Additionally or alternatively, in instances in which individually identified points and edge representations are used together, a weighted analysis may be performed in which different techniques may be weighted differently. For example, transition-edge registration determinations that are based on the pupil edge representation may be weighted differently than registration determinations based on scleral edge representations. In these and other embodiments, both of those registration determinations may be weighted differently than point-based registrations (e.g., registration determinations that are based on individual iris points).

In some embodiments, the weighting may be based on a heuristic analysis. Additionally or alternatively, the weighting may be based on specific factors corresponding to individual scenarios. For example, in instances in which the eye is fully dilated in one or more of the first image or the second image, individual iris point registration may be weighted lower than in instance in which the pupil is less dilated.

Modifications, additions, or omissions may be made to the method 200 without departing from the scope of the present disclosure. For example, the operations of method 200 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

Additionally or alternatively, in some embodiments the method 200 may include one or more adjustment operations related to an ophthalmic surgical system. For example, in some embodiments, an orientation of the eye with respect to the ophthalmic surgical system may be included in or determined based on the alignment parameters. For instance, an orientation of the eye with respect to one or more laser beams generated by one or more lasers of the ophthalmic surgical system may be determined based on or included in the alignment parameters and based on a known positional relationship between the camera used to capture the second image and the laser beams. Such a determination may be performed using any suitable technique.

In these and other embodiments, the one or more adjustment operations may include adjusting a position of the ophthalmic surgical system (e.g., adjusting positioning of the laser beams) based on the alignment parameters. For example, a particular portion of the eye may be designated for treatment (e.g., based on the first image) and the alignment parameters may be used to orient laser beams with respect to such portion to allow corresponding laser-based treatment tasks to be performed with respect to the particular portion. In some embodiments, the adjusting of the orientation may include physically moving the positions of the laser beams—e.g., via one or more actuators or motors that move the laser itself and/or by adjusting one or more laser parameters that adjust the location of the laser beam without moving the whole laser (e.g., via adjustment of mirrors, etc.). Additionally or alternatively, the adjusting of the orientation may include changing the pattern in which the laser is programmed to treat the eye such that the treatment pattern is rotated in software settings of the laser to adjust the pattern in which the laser will be fired. Additionally or alternatively, the adjusting of the orientation may include changing a pattern of the laser beams. In these and other embodiments, the adjustment of the orientation may include adjusting the position of the eye (e.g., by adjusting an orientation of a bed on which the patient is laying). In the present disclosure, reference to a “laser beam” may include any suitable beam of light that may be generated by a laser. Such light beams may be continuous or pulsed.

FIG. 3A is a flow diagram illustrating a process 300 for identifying a transition-edge portion (also referred to as an “edge portion”) corresponding to a transition edge of an eye, in accordance with one or more embodiments of the present disclosure. One or more operations of the process 300 may be performed by any suitable system, apparatus, or device such as, for example, the system 100 of FIG. 1, an ophthalmic surgical system such as that described with respect to FIG. 6, and/or a computing system such as that described with respect to FIG. 7. In addition, in some embodiments, one or more of the operations of the process 300 may be used with respect to block 202 of FIG. 2 to determine the first transition-edge portion discussed therewith and/or one or more of the operations of the process 300 may be used with respect to block 206 of FIG. 2 to determine the second transition-edge portion discussed therewith. The process 300 may be performed with respect to an image that depicts an eye, such as the first image 106a and/or the second image 106b discussed with respect to FIG. 1.

In some embodiments, the process 300 may include an operation 302 corresponding to establishing a polar coordinate system with respect to the image. For example, in some embodiments, the center of the pupil may be identified in the image using any suitable technique. Additionally or alternatively, the center of the pupil may be used as the origin and a horizontal line running through the origin may be used as the x-axis. In these and other embodiments, individual locations or points within the image (e.g., pixels) are indicated with a radial coordinate “r” indicating a distance of the respective locations from the origin (e.g., center of the pupil) and an angular coordinate corresponding to the angle between the x-axis and a line segment between the origin and respective locations, which may also be referred to as an “angular location” in the present disclosure.

For example, FIG. 3B illustrates an example image 350 of an eye. A center 352 of the pupil may be used as the origin and a line 354 may be used as the x-axis for the polar coordinate system. For instance, a point 356, located a distance “d” from the center 352 and located along a line segment that has an angular location 45° offset from the line 354 may have an “r” coordinate of “d” and an angular coordinate of “θ=45°”, which may be expressed as “(d, θ)”. Further, FIG. 3B illustrates various slices of the image 350 that are slices between certain degree ranges of “θ”. For example, a slice 358 may be a 45° slice between “θ” values of 0° to 45°.

As discussed in further detail, the polar coordinates may be used in various aspects of identification of the edge portion and/or defining the edge portion. Further, the polar coordinates may be used with respect to other operations corresponding to eye registration such as those described with respect to FIGS. 4A, 4B, 5A, and 5B.

Returning to FIG. 3A, in some embodiments the process 300 may include an operation 304 corresponding to identifying edge-points of an image of that correspond to a target transition edge of the eye. In some embodiments, the operation 304 may include detecting, as depicted in the image, a target portion of the eye that corresponds to the target transition edge. For example, in instances in which the target transition edge is the outer edge of the pupil, the target portion may be the pupil, which may be generally identified using any suitable technique. Additionally or alternatively, in instances in which the target transition edge is the inner edge of the sclera, the target portion may be the sclera, which may also be generally identified using any suitable technique.

In these and other embodiments, the edge-points of the target transition edge may be identified using pixel data. For example, intensity data of pixels that correspond to the pupil may have significantly lower intensity values than pixels that correspond to the iris. Therefore, points in the image (e.g., pixels) that correspond to the outer edge of the pupil (“pupil-edge points”) may be identified based on abrupt changes from lower intensity values (e.g., of corresponding pixels) corresponding to the area of the image generally identified as corresponding to the pupil to significantly higher intensity values, which may correspond to the area of the image that may be generally identified as corresponding to the iris.

For example, the intensity values of pixels that are identified as corresponding to the pupil (“pupil pixels”) may be compared against those of neighboring pixels (e.g. pixels that are within a certain number of pixels from an individual pixel (e.g., within 3 pixels)). The comparisons may be made to determine intensity value differences (“difference values”) between the individual pixel and its neighboring pixels such that a set of intensity value differences may be determined for each individual pupil pixel. Each difference value in each set may correspond to the difference in intensity value between that of the individual pupil pixel to which the set corresponds and one of its neighboring pixels.

For example, a pupil pixel “P0” may have an intensity value “10”. In addition, in the present example pixels that are immediately adjacent to “P0” may be considered neighbor pixels. Further, “P0” may have square shape such that “P0” may have four pixels immediately adjacent thereto (“P1”, “P2”, “P3”, and “P4”). “P1” may have an intensity value “i1”, “P2” may have an intensity value “i2”, “P3” may have an intensity value “i3”, and “P4” may have an intensity value “i4”. The set of difference values for “P0” in the present example may accordingly include a first difference value “DV1” (e.g., the absolute value of “i0−i1”), a second difference value “DV2” (e.g., the absolute value of “i0−i2”), a third difference value “DV3” (e.g., the absolute value of “i0−i3”), and a fourth difference value “DV4” (e.g., the absolute value of “i0−i4”). The set of difference values for “P0” may be expressed as [DV1, DV2, DV3, and DV4].

In these and other embodiments, the points (e.g., pixels) whose set of difference values satisfies one or more criteria may be identified as pupil-edge points. In some embodiments, the criteria for selection of a pupil-edge point from the pupil pixels may include selecting, as the pupil-edge points, the pupil pixels that have the highest intensity value difference determinations corresponding therewith as compared to the other pupil pixels that have the same angle coordinate or that have angle coordinates within a specified range of the particular angle coordinate of such pupil pixel.

For example, the image 350 of FIG. 3B may include a line segment 360 having an angular location that corresponds to “θ” equaling 90°. In these and other embodiments, a pupil-edge point 362 may be determined with respect to the line segment 360 based on different sets of difference values that may respectively correspond to different pupil points that are along the line segment 360 (referred to as 90°-pupil points).

For instance, the highest difference value among the sets of difference values corresponding to the 90°-pupil points may be determined. In these and other embodiments, the 90°-pupil point corresponding to the highest difference value may be identified as the pupil-edge point 362. Additionally or alternatively, an average difference value may be determined for individual sets of difference values corresponding to the 90°-pupil points. In these and other embodiments, the 90°-pupil point corresponding to the set with the highest average difference value may be selected as the pupil-edge point 362.

Additionally or alternatively, rather than looking at difference values corresponding to individual line segments, sets of difference values corresponding to larger slices of the pupil (e.g., one degree slices of the pupil) may be used to determine pupil-edge points. In these and other embodiments, rather than the sets of difference values including individual difference values corresponding to all of the surrounding pixels, the sets of difference values may only correspond to neighboring pixels that radially extend from the center of the pupil (e.g., pixels that are along a same line segment corresponding to a same value of “θ”).

The number of pupil-edge points and different angular positions of the pupil-edge points that may be identified may be based on a target resolution for the pupil-edge points. For example, a first target resolution of three hundred and sixty pupil-edge points for the circumference of the pupil may be such that a different pupil-edge point may be identified for each one-degree increment. As another example, a second target resolution of seven hundred and twenty pupil-edge points for the circumference of the pupil may be such that a different pupil-edge point may be identified for each half-degree increment.

The target resolution may be based on a variety of factors. For example, in some embodiments, the target resolution may be based on the overall resolution of the image such that the target resolution of the pupil-edge points may be the same as the resolution of the image in general—e.g., the pupil-edge points may each correspond to a pixel, and adjacent pupil-edge points of any particular pupil-edge point may correspond to adjacent pixels of the pixel corresponding to the particular pupil-edge point. Additionally or alternatively, the target resolution of the pupil-edge points may be set by a user and/or based on a heuristic analysis with respect to a relationship between the target resolution and registration accuracy. Additionally or alternatively, the target resolution may be based on computing resources that may be available for registration in that registration based on a smaller target resolution may use fewer computing resources.

The principles associated with the examples given above with respect to identifying pupil-edge points may also be applicable for the identification of scleral-edge points as well. For example, intensity data of pixels that correspond to the sclera may have significantly higher intensity values than pixels that correspond to the iris. Therefore, points in the image that correspond to the inner edge of the sclera (“scleral-edge points”) may be identified based on abrupt changes from higher intensity values corresponding to the area of the image generally identified as corresponding to the sclera to significantly lower intensity values, which may correspond to the area of the image that may be generally identified as corresponding to the iris. In these and other embodiments, difference values corresponding to sclera points that are determined in an analogous manner as those described above with respect to the pupil points may be used to identify the scleral-edge points in a similar or analogous manner.

In some embodiments, the process 300 may include an operation 306. The operation 306 may include identifying a transition-edge line (“edge line”) based on the edge points identified at operation 304. In some embodiments, the edge line may be a pupil-edge line that is identified based on pupil-edge points. Additionally or alternatively, the edge line may be a scleral-edge line that is identified based on scleral-edge points. In these and other embodiments, the operation 306 may include identifying only the pupil-edge line, only the scleral-edge line, or both the pupil-edge line and the scleral-edge line.

In some embodiments, the pupil-edge line may correspond to the full circumference of the pupil—e.g., a full 360° radial portion that corresponds to all values of “θ” between 0° and 360°. Additionally or alternatively, the pupil-edge line may include only a portion of the circumference of the pupil edge—e.g., a radial portion or slice that corresponds to a particular range or span of angles. In these and other embodiments, the scleral-edge line may correspond to the full circumference of the scleral edge—e.g., a full 360° radial portion. Additionally or alternatively, the pupil-edge line may include only a portion of the scleral edge—e.g., a radial portion or slice that corresponds to a particular range of angular location values. By way of example, FIG. 3B illustrates an example pupil-edge line 366 that extends around the full circumference of the pupil of the eye depicted in the image 350.

In some embodiments, the operation 306 may include a block 308 at which a plot of the edge points may be used as the edge line. For instance, the pupil-edge line may be generated by forming a line connecting pupil-edge points. Additionally or alternatively, the scleral-edge line may be generate by forming a line connecting the scleral-edge points.

In these and other embodiments, the operation 306 may include a block 310 at which an ellipse may be used to identify one or more of the edge lines. For example, in some embodiments, a first ellipse may be fitted to the pupil-edge lines and at least a portion of the line that forms the circumference of the first ellipse may be used as the pupil-edge line. Additionally or alternatively, a second ellipse may be fitted to the scleral-edge lines and at least a portion of the line that forms the circumference of the second ellipse may be used as the scleral edge line.

In these and other embodiments, the operation 306 may include a block 312 at which a filtered plot of edge points may be used as a corresponding edge line. For example, in some embodiments, the plot of pupil-edge points may be passed through a low-pass filter, which may smooth out the plot and be used to generate a filtered plot of the pupil-edge points. For example, the low-pass filter may be a Gaussian filter with a standard deviation between 1° and 5° (e.g., a Gaussian filter with a 2° standard deviation). In these and other embodiments, the filtered plot may be used as the pupil-edge line. Additionally or alternatively, a similar process may be used to obtain a filtered plot of the scleral-edge points that may be used as the scleral-edge line.

In some embodiments, the process 300 may include an operation 314. The operation 314 may include identifying an edge portion based on the edge line that may be identified at operation 306. In general, the edge portion may be a portion of the corresponding image that may include the edge line. Additionally or alternatively, the edge portion may include an area around the edge line.

In some embodiments, the edge portion may be a pupil-edge portion that is identified based on the pupil-edge line. Additionally or alternatively, the edge line may be a scleral-edge portion that is identified based on the scleral-edge line. In these and other embodiments, the operation 306 may include identifying only the pupil-edge portion, only the scleral-edge portion, or both the pupil-edge portion and the scleral-edge portion.

In some embodiments, the pupil-edge portion may correspond to the full circumference of the pupil—e.g., a full 360° radial portion that corresponds to all values of “θ” between 0° and 360°. Additionally or alternatively, the pupil-edge portion may include only a portion of the circumference of the pupil—e.g., a radial portion or slice that corresponds to a particular range of angular location values. In these and other embodiments, the scleral-edge portion may correspond to the full circumference of the scleral edge—e.g., a full 360° radial portion. Additionally or alternatively, the scleral-edge portion may include only a portion of the scleral edge—e.g., a radial portion or slice that corresponds to a particular range of angular location values.

In some embodiments, the operation 314 may include a block 316 by which the edge line(s) identified at operation 306 may be used as a corresponding edge portion. For instance, the pupil-edge line may be used as the pupil-edge portion in some embodiments. In these and other embodiments, the scleral-edge line may be used as the scleral-edge portion.

Additionally or alternatively, in some embodiments, the operation 314 may include a block 318. At block 318, the edge portion may be identified by selecting a portion that radially extends away from the corresponding edge line based on a threshold distance. For example, in some embodiments, the pupil-edge portion may be identified by radially extending away from the pupil-edge line towards an outer portion of the eye by the threshold distance. Additionally or alternatively, in some embodiments, the scleral-edge portion may be identified by radially extending away from the scleral-edge line towards an inner portion of the eye by the threshold distance.

By way of example, FIG. 3B illustrates an example pupil-edge portion 368 that extends away from the pupil-edge line 366 to an outer edge 370 by a particular threshold distance. The pupil-edge line 366 may be considered an inner edge of the pupil-edge portion 368 in this example.

Although the examples given use the transition-edge lines as either inner edges (e.g., pupil-edge lines) or outer edges (e.g., scleral-edge lines) of the edge portions, these are merely given as examples. For example, in some embodiments, the edge portions may be identified by extending away from the edge lines in both directions (e.g., both towards the center 352 and towards the sclera) by a particular threshold distance. In some embodiments, the particular threshold distance may be the same in both directions. Additionally or alternatively, the particular threshold distance may vary for the two directions.

In some embodiments the threshold distance for the pupil-edge portion may be the same as that used for the scleral-edge portion. Additionally or alternatively, the threshold distance for the pupil-edge portion may be different from that used for the scleral-edge portion.

In some embodiments, the threshold distance may be a number of pixels away from the corresponding edge line. In these and other embodiments, the number of pixels may be based on the resolution of the corresponding image. Additionally or alternatively, the threshold distance may be a certain value corresponding to a certain distance with respect to the eye itself e.g., a distance in the corresponding image that corresponds to a certain number of millimeters away from the corresponding edge line, a number of pixels, etc. Additionally or alternatively, the threshold distance for the pupil-edge portion may be based on a ratio of the threshold distance with respect to the diameter of the pupil, a major axis of the pupil, a minor axis of the pupil, and/or or the length (e.g., the circumference) of the pupil-edge line. In these and other embodiments, the threshold distance for the scleral-edge portion may be based on a ratio of the threshold distance with respect to the diameter of the portion of the eye that excludes the sclera, a major axis of the portion of the eye that excludes the sclera, a minor axis of the portion of the eye that excludes the sclera, and/or the length (e.g., circumference) of the scleral-edge line.

In some embodiments, the threshold distance may be determined based on a heuristic analysis corresponding to an effect of an accuracy of the alignment registration with respect to size of the threshold distance. Additionally or alternatively, the threshold distance may be set by a user.

In some embodiments, one or more portions of the corresponding image may be cropped out based on the edge portion. For example, referring to FIG. 3B, in some embodiments, the inner pupil portion of the pupil that is radially inward from the pupil-edge line 366 may be cropped out. Additionally or alternatively, the outer eye portion of the eye that is radially outward from the outer edge 370 may also be cropped out such that only the pupil-edge portion 368 may remain.

By way of illustration, FIG. 3C illustrates the pupil-edge portion 368 following the cropping. In FIG. 3C, the pupil-edge portion 368 is illustrated as a radial strip in which the x-axis corresponds to the angular location polar coordinate values of “θ” and the y-axis corresponds to distance from the center of the pupil.

In some embodiments, a similar type of cropping may be performed with respect to a scleral-edge portion. In instances in which both a pupil-edge portion and a scleral-edge portion are identified, the inner pupil portion may be cropped out and the iris portion between the pupil-edge portion and the scleral-edge portion may also be cropped out.

The process 300 may accordingly be used to identify one or more transition-edge portions of an image that correspond to a transition edge of an eye depicted in the image. Modifications, additions, or omissions may be made to the process 300 without departing from the scope of the present disclosure. For example, the operations of process 300 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

FIG. 4A is a flow diagram illustrating an example process 400 for determining a transition-edge representation, in accordance with one or more embodiments of the present disclosure. One or more operations of the process 400 may be performed by any suitable system, apparatus, or device such as, for example, the system 100 of FIG. 1, an ophthalmic surgical system such as that described with respect to FIG. 6 of the present disclosure, and/or a computing system such as that described with respect to FIG. 7 of the present disclosure. In addition, in some embodiments, one or more of the operations of the process 400 may be used with respect to block 204 of FIG. 2 to determine the first pupil-edge representation discussed therewith and/or one or more of the operations of the process 400 may be used with respect to block 208 of FIG. 2 to determine the second pupil-edge representation discussed therewith discussed therewith. The process 400 may be performed with respect to an image that depicts an eye, such as the first image 106a and/or the second image 106b discussed with respect to FIG. 1.

Additionally or alternatively, the process 400 may be performed with respect to a transition edge portion (“edge portion”) identified by the process 300. For example, the process 400 may be performed with respect to a pupil-edge portion and/or a scleral-edge portion.

In some embodiments, the process 400 may include an operation 402 corresponding to dividing the edge portion into multiple sub-portions. For example, in some embodiments, the operation 402 may include a block 404 at which the edge portion may be divided into slices extending radially outward from an inner edge of the edge portion to an outer edge of the edge portion, in which individual slices are used respectively used as individual sub-portions.

The slices may correspond to one or more values of “θ” in some embodiments. For example, in some embodiments, the slices may each correspond to a line segment that corresponds to an angular location corresponding to a single value of “θ”. For instance, referring to FIG. 3C, a particular slice of the pupil-edge portion 368 may be a line segment 372 between pupil-edge line 366 and the outer edge 370 at “θ=45°”. Additionally or alternatively, the slices may each correspond to an angular location that is an area corresponding to a range of values of “θ”. For example, an individual slice may be a slice with an angular width of a particular number of degrees (e.g., a 1° width, a 2° width, a 5° width, etc.).

In these and other embodiments, the number of slices may vary. For example, the number of slices may be based on a target resolution corresponding to the transition-edge representation that may be generated based on the slices. For example, a first target resolution of three hundred and sixty values for a transition-edge representation corresponding to a full circumference may be such that a different slice may be identified for each one-degree increment of the corresponding edge portion. For example, a second target resolution of seven hundred and twenty values for the transition-edge representation corresponding to a full circumference may be such that a different slice may be identified for each half-degree increment of the corresponding edge portion.

The target resolution may be based on a variety of factors. For example, in some embodiments, the target resolution may be based on the overall resolution of the image such that the target resolution of the slices may be the same as the resolution of the image in general—e.g., the number of slices may correspond to the number of pixels along the angular locations of the edge portion. For example, referring to FIG. 3C, the resolution may be the number of pixels that extend between 0° and 360° along the x-axis. Additionally or alternatively, the target resolution of the slices may be set by a user and/or based on a heuristic analysis with respect to a relationship between the target resolution and registration accuracy. In these and other embodiments, the target resolution may be based on computing resources that may be available for registration in that registration based on a smaller target resolution may use fewer computing resources.

In some embodiments, the process 400 may include an operation 406. At operation 406, the transition-edge representation may be determined based on pixel information corresponding to the sub-portions identified at operation 402. In these and other embodiments, the pixel information may include intensity values corresponding to the different sub-portions.

For example, in some embodiments, the operation 406 may include a block 408. At block 408 an aggregate intensity value may be determined for each sub-portion. The aggregate intensity value may include a value that represents an aggregation of the intensity values that correspond to the respective sub-portions. For example, referring to FIG. 3C, an aggregate intensity value for the line segment 372 may be based on the different intensity values corresponding to points that lie on the line segment 372. As another example, an aggregate intensity value for a sub-portion that corresponds to a range of angular values (e.g., a certain angular width), the aggregate intensity value may be based on the different intensity values of the points that are within such angular slice.

In some embodiments, the aggregate intensity value may include an average of the intensity values of the corresponding sub-portion. Additionally or alternatively, the aggregate intensity value may include a sum of the intensity values or a product of the intensity values.

Additionally or alternatively, the individual intensity values that are used in determining the aggregated intensity values may be weighted such that the aggregate intensity values may be weighted values. For example, intensity values that correspond to locations that are closer to the transition-edge line used to generate the edge portion may be weighted higher than intensity values that are further away, or vice versa. The weighting may be based on a heuristic analysis in some embodiments.

In some embodiments, one or more filtering operations may be performed with respect to the determined aggregate intensity values. For example, in some embodiments, high pass filtering may be applied to the plot of the aggregate intensity values. In these and other embodiments, the filtering may be used to mask out bright reflections, which may remove or reduce illumination effects in some embodiments. In these and other embodiments, the filtered plot may be used as the transition-edge representation.

For example, in some embodiments a low-pass Gaussian filter with a standard deviation on the aggregate intensity values between 1° and 10° (e.g., a Gaussian filter with a 2.5° standard deviation) may be applied to the aggregate intensity values to obtain a first filtered plot. The first filtered plot may then be subtracted from the unfiltered plot of the aggregate intensity values to obtain a second filtered plot that may represent the high pass filtering. The second filtered plot may be used as the transition edge representation in some embodiments.

In some embodiments, the operation 406 may include a block 410. At the block 410 the transition-edge representation may be determined based on the aggregate intensity values. In some embodiments, the filtered aggregate intensity values may be used. Additionally or alternatively, non-filtered aggregate intensity values may be used. General reference to the aggregate intensity values may refer to generically aggregate intensity values that may or may not have been filtered.

For example, in some embodiments, the aggregate intensity values for the sub-portions may be used as values for the transition-edge representation. For instance, as indicated above, each aggregate intensity value may correspond to a particular angular location (e.g., a line segment at a particular angular location of or angular width.) Further, each aggregate intensity value may be plotted based on its corresponding angular location. The plot of the aggregate intensity values may accordingly indicate a representation of the transition edge along the angular locations for which the plot corresponds.

For example, FIG. 4B illustrates an example graph 450 of a pupil-edge representation 452 that may correspond to the pupil-edge portion 368 of FIG. 3C and that may represent the outer edge of the pupil of the image 350. The various y-values along the different angular locations represented in the x-axis of FIG. 4B may represent the aggregate intensity values that may be determined for the different sub-portions that correspond to the different angular locations. The plot of such values may be used to form the pupil-edge representation 452.

The process 400 may accordingly be used to identify one or more transition-edge representations. Modifications, additions, or omissions may be made to the process 400 without departing from the scope of the present disclosure. For example, the operations of process 400 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

FIG. 5A is a flow diagram illustrating an example process 500 for determining an alignment registration, in accordance with one or more embodiments of the present disclosure. One or more operations of the process 500 may be performed by any suitable system, apparatus, or device such as, for example, the system 100 of FIG. 1, an ophthalmic surgical system such as that described with respect to FIG. 6 of the present disclosure, and/or a computing system such as that described with respect to FIG. 7 of the present disclosure. In addition, in some embodiments, one or more of the operations of the process 500 may be used with respect to block 210 of FIG. 2

The process 500 may be performed with respect to a first transition-edge representation and a second transition-edge representation corresponding to two different images but a same portion of an eye. For example, the process 500 may be performed with respect to a first pupil-edge representation corresponding to a first image (e.g., the first image 106a of FIG. 1) and with respect to a second pupil-edge representation corresponding to a second image (e.g., the second image 106b of FIG. 1). Additionally or alternatively, the process 500 may be performed with respect to a first scleral-edge representation corresponding to the first and with respect to a second pupil-edge representation corresponding to the second image. In these and other embodiments, the first transition-edge representation and/or the second transition-edge representation may be obtained based on one or more operations described with respect to the process 300 of FIG. 3A and/or the process 400 of FIG. 4A.

In some embodiments, the process 500 may include an operation 502 corresponding to determining a correlation distribution between the first transition-edge representation and the second transition-edge representation. The correlation distribution may indicate the correlations between different parts of the first transition-edge representation and the second transition-edge representation. For example, in some embodiments, the correlation distribution may indicate a correlation between each first portion of multiple first portions of the first transition-edge representation and each second portion of multiple second portions of the second transition-edge representation. In these and other embodiments, the correlation distribution may be represented using a heatmap that is based on the correlations.

For instance, in some embodiments, the operation 502 may include a block 504. At block 504 a template may be extracted from the first transition-edge representation. The template may be a certain portion of the first transition-edge representation that corresponds to a particular angular width and that may correspond to a particular angular location of the first transition-edge representation. For example, FIG. 5B illustrates an example graph 550 of a first pupil-edge representation 552, which may be the same or analogous to the graph 450 of the pupil-edge representation 452 of FIG. 4B. FIG. 5B also illustrates an example template 554. The template 554 may correspond to an angular location of “θ1” and may have a width of “n” degrees between “θ1” and “θ2” in which “θ1+n=θ2”.

Additionally or alternatively, in some embodiments, the operation 502 may include a block 506. At block 506, the template that is extracted at block 504 may be compared against the second transition-edge representation at multiple angular locations along the second transition-edge representation. For example, the values of the first transition-edge representation that are included in the template may be aligned with the values of the second transition-edge representation starting at a particular angular location. Such an operation may be referred to as aligning the template with a comparison portion of the second transition-edge representation.

For example, FIG. 5B includes a plot 556 of a second pupil-edge representation 558. Further, FIG. 5B illustrates the template 554 aligned with a comparison portion 560 of the second pupil-edge representation 558. In particular, the comparison portion 560 may begin at angular location “φ1” and may be the same angular width as the template 554 such that the comparison portion 560 may end at angular location “φ2”, which may be equal to “φ1+n”. In the present disclosure, reference to a comparison portion corresponding to a particular angular location may refer to the lowest angular location of such comparison portion. For example, the comparison portion 560 may be referred to as corresponding to the angular location of “φ1”.

The template 554 may be aligned with the comparison portion 560. For example, the value of the first pupil-edge representation 552 that corresponds to the angular location “θ1” in the graph 550 may be aligned with the value of the second pupil-edge representation 558 that corresponds to the angular location “φ1” in the graph 556. Similarly, the value of the first pupil-edge representation 552 that corresponds to the angular location “θ1+1°” in the graph 550 may be aligned with the value of the second pupil-edge representation 558 that corresponds to the angular location “φ1+1°” in the graph 556 and so forth for different angular locations along the comparison portion 560. Note that the illustration of superimposing the template 554 with the comparison portion 560 is merely to help describe concepts discussed herein and is not meant to be limiting.

Returning to FIG. 5A, in some embodiments, a comparison between the template and the comparison portion (e.g., between values respectively corresponding to the template and the comparison portion) may be performed to determine similarity between the template and the comparison portion. In some embodiments, the similarity may be determined by performing a correlation determination between the template and the comparison portion. The correlation determination may include any suitable correlation determination. For example, in some embodiments, the correlation determination may include determining a normalized cross-correlation between the template and the comparison portion.

In some embodiments, the operations of blocks 504 and 506 may be performed with respect to multiple different templates and multiple different comparison portions. For example, referring to FIG. 5B, a first correlation may be determined with respect to the template 554 and a first comparison portion that begins at the angular location of 0° along the graph 556, further a second correlation may be determined with respect to the template 554 and a second comparison portion that begins at the angular location of 1° along the graph 556, a third correlation may be determined with respect to the template 554 and a third comparison portion that begins at the angular location of 2° along the graph 556, and so forth until the template 554 has been compared against a series of different comparison portions that incrementally cover all of the second pupil-edge representation 558 from 0° to 360°. Further, similar operations may be performed with respect to a first template 554 that begins at the angular location of 0° along the graph 550, a second template that begins at the angular location of 1° along the graph 550, a third template that begins at the angular location of 2° along the graph 550, and so forth until different templates have been generated that incrementally cover all of the first pupil-edge representation 552 from 0° to 360°. In some embodiments, the comparison may be limited to certain angular locations along the graph 556, such as +/−5° (e.g., from 355° to 5°), +/−7° (e.g., from 353° to 7°), +/−10° (e.g., from 350° to 10°), +/−12° (e.g., from 348° to 12°), +/−15° (e.g., from 345° to 15°), +/−18° (e.g., from 342° to 18°), +/−20° (e.g., from 330° to 20°), or any other range.

The performance of the operations of blocks 504 and 506 with respect to multiple different templates and multiple different comparison portions may be used to generate the correlation distribution—e.g., heat map. The correlation distribution may indicate which first values at first angular locations of the first transition-edge representation may align with second values at second angular locations of the second transition-edge representation. In some embodiments, the correlation distribution may be represented using a heatmap.

In some embodiments, the process 500 may include an operation 508 corresponding to determining an alignment registration based on the correlation distribution determined with respect to operation 502. In general, the correlation distribution may be used to an angular shift (e.g., amount of rotation) that may be made to the first image with respect to the second image to align the eyes that are depicted therein.

In some embodiments, the operation 508 may include a block 510 corresponding to dividing the correlation distribution into multiple sub-portions. For example, in some embodiments, the correlation distribution may be divided into slices. The slices may correspond to one or more angular offset values in some embodiments.

For example, in some embodiments, the slices may each correspond to a line segment that corresponds to a particular angular offset value. Additionally or alternatively, the slices may each correspond to an angular offset that is an area corresponding to a range of angular offsets. For example, an individual slice may be a slice with an angular width of a particular number of degrees (e.g., a 1° width, a 2° width, a 5° width, etc.).

In these and other embodiments, the number of slices may vary. For example, the number of slices may be based on a target resolution. For example, a first target resolution of three hundred and sixty values may be such that a different slice may be identified for each one-degree increment of the corresponding edge portion. For example, a second target resolution of seven hundred and twenty values may be such that a different slice may be identified for each half-degree increment of the corresponding edge portion.

The target resolution may be based on a variety of factors. For example, in some embodiments, the target resolution may be based on the overall resolution of the correlation distribution such that the target resolution of the slices may be the same as the resolution of the correlation distribution—e.g., the number of slices may correspond to the number of data points along the angular offset axis of the correlation distribution. Additionally or alternatively, the target resolution of the slices may be set by a user and/or based on a heuristic analysis with respect to a relationship between the target resolution and registration accuracy. In these and other embodiments, the target resolution may be based on computing resources that may be available for registration in that registration based on a smaller target resolution may use fewer computing resources.

In some embodiments, the operation 508 may include a block 512. At block 512 an aggregate correlation value may be determined for each sub-portion. The aggregate correlation value may include a value that represents an aggregation of the correlation values that correspond to the respective sub-portions. As another example, an aggregate intensity value for a sub-portion that corresponds to a range of angular values (e.g., a certain angular width), the aggregate correlation value may be based on the different intensity values of the points that are within such angular slice.

In some embodiments, the aggregate correlation value may include an average of the correlation values corresponding to the corresponding sub-portion. In these and other embodiments, the aggregate correlation value may include a sum of the intensity values or a product of the intensity values. Additionally or alternatively, the individual correlation values that are used in determining the aggregated correlation values may be weighted such that the aggregate correlation values may be weighted values. For example, in some instances some a priori knowledge may be present with respect to possible eye orientations and corresponding offset angles. Such knowledge may be used to weigh correlation values corresponding to such offset angles.

In some embodiments, the operation 508 may include a block 514. At block 514, a correlation representation may be determined based on the aggregate correlation values for the sub-portions of the correlation distribution. For example, as indicated above, each aggregate correlation value may correspond to a particular angular offset (e.g., a line segment at a particular angular location of or angular width.) In these and other embodiments, each aggregate correlation value may be plotted based on its corresponding angular offset. The plot of the aggregate correlation values may accordingly indicate a representation of the different correlations along the angular offset values for which the plot corresponds.

For example, FIG. 5D illustrates an example graph 576 of a correlation representation 578 that may correspond to a correlation distribution. The various y-values along the different angular offset values represented in the x-axis of FIG. 5D may include the aggregate correlation values that may be determined for the different sub-portions that correspond to the different angular locations. The plot of such values may be used to form the correlation representation 578.

In some embodiments, the operation 508 may include a block 516. At block 516 the alignment registration may be determined based on the correlation representation. For example, the angular offset value that corresponds to the highest values of the correlation representation may indicate the amount of angular offset between the eye as depicted in the first image and the eye as depicted in the second image.

As such, in some embodiments, a maximum corresponding to the y-axis of the correlation representation may be identified. In these and other embodiments, the angular offset value that corresponds to the maximum may be determined to be the amount of angular offset (e.g., cyclotorsion rotation) between the first image and the second image and may be included in the alignment registration between the first image and the second image.

The maximum may be found in any suitable manner. For example, in some embodiments, the maximum may be the absolute maximum value of the correlation representation. In such embodiments, the angular offset value corresponding to the absolute maximum value may be included in the alignment registration.

By way of example, FIG. 5D illustrates a graph 580 of a portion 582 of the correlation representation 578 of the graph 576. The portion 582 corresponds to the portion of the correlation representation that has the highest correlation value (e.g., the absolute maximum value). In particular, a point 584 may have the absolute maximum value of the correlation representation 578. Further, the angular offset value of the point 574 may be 0.5°. As such, in some embodiments with respect to this particular example, the alignment registration (e.g., cyclotorsion registration) may include an indication that the eye in the second image is rotated with respect to the eye in first image by 0.5°.

Additionally or alternatively, the maximum may correspond to a range of correlation distribution values that are within a portion of the correlation distribution that is determined to include the absolute maximum. In these and other embodiments, the angular offset value that may be included with the alignment registration may include an aggregate offset value corresponding to such a portion. In some embodiments, the aggregate offset value may be an average of the offset values included in the portion. In these and other embodiments, the aggregate offset value may be based only on offset values that correspond to correlation representation values that satisfy a particular threshold (e.g., the top 1%, 5%, 10%, etc. of correlation representation values within the portion). Additionally or alternatively, the aggregate offset value may be a weighted average. For example, offset values closer to the offset value corresponding to the absolute maximum offset value may be weighted higher than those that are further away.

Referring again to FIG. 5D, by way of example, an aggregate offset value may be determined with respect to the portion 582, in any suitable manner such as those described above. In these and other embodiments, such aggregate offset value may be used as the angular offset between the eye in the first image and the eye in the second image.

The process 500 may accordingly be used to identify an alignment registration between eyes depicted in two images. Modifications, additions, or omissions may be made to the process 500 and the accompanying FIGS. 5A-5D without departing from the scope of the present disclosure. For example, the operations of process 500 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

For example, the angular width of the templates and/or the comparison portions may vary. Further, the change in angular distance between one template and an adjacent template and/or one comparison portion and an adjacent comparison portion may also vary. For example, in some embodiments, the angular distance change may be the same as the resolution of the first transition-edge representation and/or the second transition-edge representation—e.g., the angular distance change may be the same as the angular distance between adjacent points in the first transition-edge representation and/or the second transition-edge representation. Additionally or alternatively, the angular distance change may be greater than the resolution of the first transition-edge representation and/or the second transition-edge representation—e.g., the angular distance change may be greater than the angular distance between adjacent points in the first transition-edge representation and/or the second transition-edge representation.

In some embodiments, the angular width and/or angular distance change may be set by a user and/or determined based on a heuristic analysis with respect to a relationship between registration accuracy and the angular width and/or angular distance. In these and other embodiments, the angular width and/or angular distance change may be based on computing resources that may be available for registration.

Further, although the templates are described in the examples as being extracted from the first transition-edge representation and compared against different comparison portions of the second transition-edge representation, such operations may be reversed in which the templates may be extracted from the second transition-edge representation and compared against the first transition-edge representation.

In addition, the graphs are merely used as examples and are not meant to be limiting. For example, the values that correspond to the different axes of the graphs are only examples. As such, other graphs in which different values correspond to the axes may be just as applicable and are contemplated. Additionally or alternatively, no graphs may be actually generated and calculations or processing of numerical values may occur.

FIG. 6 is a block diagram of an example ophthalmic surgical system 600 (“system 600”) that may be used with and/or implement one or more embodiments of the present disclosure related to alignment registration of an eye. In general, the system 600 may be configured to perform one or more ophthalmic treatment operations with respect to a target tissue 612, which may be a lens or a portion of a lens of an eye. In some embodiments, the system 600 may include a laser 602, an optics module 604, an imaging system 606, a control module 608, and a patient interface 610.

The laser 602 may include any suitable system, apparatus, or device, configured to generate one or more laser beams that may be used to perform ophthalmic operations or tasks with respect to the target tissue 612. In some embodiments, the laser 602 may include multiple lasers that each generate an individual laser beam. Additionally or alternatively, the laser 602 may include a single laser that is configured to generate a single beam or multiple beams.

In some embodiments, the laser 602 may be configured to generate a pulsed laser beam that is pulsed at a high repetition rate at a pulse repetition rate of thousands of shots per second or higher with relatively low energy per pulse. For example, in some embodiments, the laser 602 may be a femtosecond laser that emits ultra-short pulses of light (e.g., on the order of 10\hat{ }⁻¹⁵ seconds). Such a laser may be operated to use relatively low energy per pulse to localize the tissue effect of the target tissue 612 that may be caused by laser-induced photodisruption by the beam generated by the laser 602.

The optics module 604 may include any suitable system, apparatus, or device that may be configured to focus and direct the laser beam to the target tissue 612. For example, in some embodiments, the optics module 604 may include one or more lenses and/or one or more reflectors (e.g., mirrors). Additionally or alternatively, in some embodiments, the optics module 604 may include one or more actuators that may be configured to adjust the focusing and/or the beam direction in response to a beam control signal that may be received from the control module 608. In these and other embodiments, the one or more actuators may be adjusted in response to a user input via any suitable user interface, such as discussed with respect to the computing system of FIG. 7. For example, in some embodiments, the user interface may include a touch screen, mouse, keyboard, joystick, foot pedal, game pad, game controller, etc., that may be used to provide commands for movement of laser beam (e.g., via the actuators).

The imaging system 606 may include any suitable system, apparatus, or device, that may be configured to obtain one or more images of the eye corresponding to the target tissue 612. For example, in some embodiments, the imaging system 606 may collect reflected or scattered light or sound from the target tissue 612 to capture image data corresponding to the target tissue 612.

The imaging system 606 may include one or more different types of devices and/or systems configured to capture various different types of images of the eye as corresponding to the target tissue 612. For example, in some embodiments, the imaging system 606 may include a camera configured to capture one or more camera images of the eye (e.g., such as those used to perform alignment registration as discussed in the present disclosure). Additionally or alternatively, the imaging system 606 may include an optical coherent tomography (OCT) device configured to capture OCT images of the eye. In these and other embodiments, the imaging system may include an ultrasound imaging device configured to capture ultrasound images of the eye.

The patient interface 610 may include a mount that is configured to engage with the target tissue 612 to hold the target tissue 612 in position during performance of the ophthalmic procedure. In these and other embodiments, the patient interface 610 may be configured to allow the laser beam to pass therethrough to allow for performance of the procedure via the laser beam.

The control module 608 may include any suitable system, apparatus, or device, configured to perform one or more control operations with respect to the system 600. For example, in some embodiments, the control module 608 may include code and routines configured to allow a computing system to perform one or more operations. Additionally or alternatively, the control module 608 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the control module 608 may be implemented using a combination of hardware and software. In the present disclosure, operations described as being performed by the control module 608 may include operations that the control module 608 may direct a corresponding computing system to perform. In these or other embodiments, the control module 608 may be implemented by one or more computing systems, such as that described in further detail with respect to FIG. 7 of the present disclosure.

In some embodiments, the control module 608 may be configured to control the laser 602 and/or the optics module 604. Additionally or alternatively, the control module 608 may be configured to control any number of other components of the system 600 not expressly illustrated.

Additionally or alternatively, the control module 608 may be configured to determine the placement of the laser beam and corresponding laser pulses with respect to the target tissue 612. For example, in some embodiments, the control module 608 may include a registration module such as described with respect to the registration module 102 of FIG. 1. The control module 608 may accordingly be configured in some embodiments to determine an alignment registration based on one or more images received from the imaging system 606 and one or more other diagnostic images that had been previously captured (e.g., during a diagnosis and via a different camera than that of the imaging system 606 and/or the same camera). In these and other embodiments, the control module 608 may be configured to determine the placement of the laser beam and the corresponding laser pulses based on the alignment registration.

Additionally or alternatively, the control module 608 may be configured to cause one or more adjustment operations (e.g., such as described in the present disclosure) to be performed based on the determined placement. For example, the control module 608 may control one or more actuators of the optics module 604 to adjust a location of the laser beam and corresponding laser pulses with respect to the target tissue 612. Additionally or alternatively, the control module 608 may adjust a pattern of the laser beam through adjustment of the laser 602 and/or the optics module 604 (e.g., to rotate a planned treatment according to the alignment registration). In these and other embodiments, the control module 608 may adjust one or more other actuators that may move the entire system 600 or the laser 602 itself such that the laser orientation may be moved with respect to the target tissue 612. Additionally or alternatively, the control module 608 may cause the adjustment of a support platform (e.g., bed) that the patient is lying on to adjust the orientation of the laser 602 with respect to the target tissue 612 (e.g., by controlling one or more actuators corresponding to the support platform).

Modifications, additions, or omissions may be made to FIG. 6 without departing from the scope of the present disclosure. For example, the system 600 may include more or fewer elements depending on the implementation. Further, the system 600 may be configured to perform any number of operations as compared to those explicitly described.

### Example Computing System

FIG. 7 is a block diagram of an example computing system 700 suitable for use in implementing some embodiments of the present disclosure. Computing system 700 may include an interconnect system 702 that directly or indirectly couples the following devices: memory 704, one or more central processing units (CPUs) 706, one or more graphics processing units (GPUs) 708, a communication interface 710, I/O ports 712, input/output components 714, a power supply 716, one or more presentation components 718 (e.g., display(s)), and one or more logic units 720.

Although the various blocks of FIG. 7 are shown as connected via the interconnect system 702 with lines, this is not intended to be limiting and is for clarity only. For example, in some embodiments, a presentation component 718, such as a display device, may be considered an I/O component 714 (e.g., if the display is a touch screen). As another example, the CPUs 706 and/or GPUs 708 may include memory (e.g., the memory 704 may be representative of a storage device in addition to the memory of the GPUs 708, the CPUs 706, and/or other components). In other words, the computing system of FIG. 7 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” “augmented reality system,” and/or other device or system types, as all are contemplated within the scope of the computing system of FIG. 7.

The interconnect system 702 may represent one or more links or busses, such as an address bus, a data bus, a control bus, or a combination thereof. The interconnect system 702 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 706 may be directly connected to the memory 704. Further, the CPU 706 may be directly connected to the GPU 708. Where there is direct, or point-to-point, connection between components, the interconnect system 702 may include a PCIe link to carry out the connection. In these examples, a PCI bus need not be included in the computing system 700.

The memory 704 may include any of a variety of computer-readable media. The computer-readable media may be any available media that may be accessed by the computing system 700. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 704 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computing system 700. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 706 may be configured to execute at least some of the computer-readable instructions to control one or more components of the computing system 700 to perform one or more of the methods and/or processes described herein. The CPU(s) 706 may each include one or more cores (e.g., one, two, four, eight, twenty-eight, seventy-two, etc.) that are capable of handling a multitude of software threads simultaneously. The CPU(s) 706 may include any type of processor, and may include different types of processors depending on the type of computing system 700 implemented (e.g., processors with fewer cores for mobile devices and processors with more cores for servers). For example, depending on the type of computing system 700, the processor may be an Advanced RISC Machines (ARM) processor implemented using Reduced Instruction Set Computing (RISC) or an x86 processor implemented using Complex Instruction Set Computing (CISC). The computing system 700 may include one or more CPUs 706 in addition to one or more microprocessors or supplementary co-processors, such as math co-processors.

In addition to or alternatively from the CPU(s) 706, the GPU(s) 708 may be configured to execute at least some of the computer-readable instructions to control one or more components of the computing system 700 to perform one or more of the methods and/or processes described herein. One or more of the GPU(s) 708 may be an integrated GPU (e.g., with one or more of the CPU(s) 706 and/or one or more of the GPU(s) 708 may be a discrete GPU. In embodiments, one or more of the GPU(s) 708 may be a coprocessor of one or more of the CPU(s) 706. The GPU(s) 708 may be used by the computing system 700 to render graphics (e.g., 3D graphics) or perform general purpose computations. For example, the GPU(s) 708 may be used for General-Purpose computing on GPUs (GPGPU). The GPU(s) 708 may include hundreds or thousands of cores that are capable of handling hundreds or thousands of software threads simultaneously. The GPU(s) 708 may generate pixel data for output images in response to rendering commands (e.g., rendering commands from the CPU(s) 706 received via a host interface). The GPU(s) 708 may include graphics memory, such as display memory, for storing pixel data or any other suitable data, such as GPGPU data. The display memory may be included as part of the memory 704. The GPU(s) 708 may include two or more GPUs operating in parallel (e.g., via a link). The link may directly connect the GPUs (e.g., using NVLINK) or may connect the GPUs through a switch (e.g., using NVSwitch). When combined together, each GPU 708 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 706 and/or the GPU(s) 708, the logic unit(s) 720 may be configured to execute at least some of the computer-readable instructions to control one or more components of the computing system 700 to perform one or more of the methods and/or processes described herein. In embodiments, the CPU(s) 706, the GPU(s) 708, and/or the logic unit(s) 720 may discretely or jointly perform any combination of the methods, processes and/or portions thereof. One or more of the logic units 720 may be part of and/or integrated in one or more of the CPU(s) 706 and/or the GPU(s) 708 and/or one or more of the logic units 720 may be discrete components or otherwise external to the CPU(s) 706 and/or the GPU(s) 708. In embodiments, one or more of the logic units 720 may be a coprocessor of one or more of the CPU(s) 706 and/or one or more of the GPU(s) 708.

Examples of the logic unit(s) 720 include one or more processing cores and/or components thereof, such as Tensor Cores (TCs), Tensor Processing Units (TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), I/O elements, peripheral component interconnect (PCI) or peripheral component interconnect express (PCIe) elements, and/or the like.

The communication interface 710 may include one or more receivers, transmitters, and/or transceivers that enable the computing system 700 to communicate with other computing systems via an electronic communication network, including wired and/or wireless communications. The communication interface 710 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The I/O ports 712 may enable the computing system 700 to be logically coupled to other devices including the I/O components 714, the presentation component(s) 718, and/or other components, some of which may be built into (e.g., integrated in) the computing system 700. Illustrative I/O components 714 include a microphone, mouse, keyboard, joystick, foot pedal, game pad, game controller, satellite dish, scanner, printer, wireless device, etc. The I/O components 714 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition associated with a display of the computing system 700. The computing system 700 may include depth cameras, such as stereoscopic camera systems, infrared camera systems, RGB camera systems, touchscreen technology, and combinations of these, for gesture detection and recognition. Additionally, the computing system 700 may include accelerometers or gyroscopes (e.g., as part of an inertia measurement unit (IMU)) that enable detection of motion. In some examples, the output of the accelerometers or gyroscopes may be used by the computing system 700 to render immersive augmented reality or virtual reality.

The power supply 716 may include a hard-wired power supply, a battery power supply, or a combination thereof. The power supply 716 may provide power to the computing system 700 to enable the components of the computing system 700 to operate.

The presentation component(s) 718 may include a display (e.g., a monitor, a touch screen, a television screen, a heads-up-display (HUD), other display types, or a combination thereof), speakers, and/or other presentation components. The presentation component(s) 718 may receive data from other components (e.g., the GPU(s) 708, the CPU(s) 706, etc.), and output the data (e.g., as an image, video, sound, etc.).

Modifications, additions, or omissions may be made to FIG. 7 without departing from the scope of the present disclosure. For example, the system 700 may include more or fewer elements depending on the implementation. Further, the system 700 may be configured to perform any number of operations as compared to those explicitly described.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

The present disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example The following is a non-limiting summary of some example implementations presented herein.

Example 1. A system comprising:
a computing system configured to cause performance of operations, the operations comprising:
   identifying a first transition-edge portion of a first image of an eye, the first transition-edge portion corresponding to a transition edge of the eye;
   determining, based on first-portion pixel data corresponding to the first transition-edge portion, a first transition-edge representation of the transition edge;
   identifying a second transition-edge portion of a second image of the eye, the second transition-edge portion corresponding to the transition edge of the eye;
   determining, based on second-portion pixel data corresponding to the second transition-edge portion, a second transition-edge representation of the transition edge; and
   determining an alignment registration between the eye as depicted in the first image and the eye as depicted in the second image based on a transition-edge based registration that is based on a comparison between the first transition-edge representation and the second transition-edge representation.

Example 2. The system of Example 1, wherein the transition edge corresponds to an outer edge of a pupil of the eye.

Example 3. The system of Example 1, wherein the transition edge corresponds to an inner edge of a sclera of the eye.

Example 4. The system of any of Examples 1-3, wherein:
the determining of the first transition-edge representation based on the first-portion pixel data is based on a plurality of first-edge intensity values determined from the first-portion pixel data; and
the determining of the second transition-edge representation based on the second-portion pixel data is based on a plurality of second-edge intensity values determined from the second-portion pixel data.

Example 5. The system of any of Example 4, wherein the first-edge intensity values include a plurality of aggregate first-edge intensity values that individually correspond to a respective first sub-portion of a plurality of first sub-portions of the first transition-edge portion, each aggregate first-edge intensity value being based on first sub-portion intensity values of its corresponding first sub-portion; and
determining the first transition-edge representation based on the plurality of aggregate first-edge intensity values.

Example 6. The system of Example 5, wherein each first sub-portion of the plurality of first sub-portions includes a slice of pixels extending radially outward from an inner edge of the first transition-edge portion to an outer edge of the first transition-edge portion.

Example 7. The system of any of Examples 5-6, wherein one or more of the first-edge intensity values are based on one or more of:
an average of the first sub-portion intensity values of their corresponding first sub-portions;
a sum of the first sub-portion intensity values of their corresponding first sub-portions; or
one or more weight values that are respectively applied to one or more of the first sub-portion intensity values.

Example 8. The system of Example 7, wherein the one or more weight values are based on a distance away from an identified center of a pupil of the eye.

Example 9. The system of any of Examples 1-8, wherein the identifying of the first transition-edge portion includes:
identifying first-edge points in the first image, the first-edge points corresponding to the transition edge;
identifying, in the first image, a first-edge line corresponding to the transition edge based on the first-edge points; and
identifying the first transition-edge portion based on the first-edge line.

Example 10. The system of Example 9, wherein the identifying of the first transition-edge portion based on the first-edge line includes identifying the first transition-edge portion based on a threshold distance radially extended away from the first-edge line.

Example 11. The system of Example 10, wherein the threshold distance is based on one or more of:
a number of pixels in the first image away from the first-edge line;
a ratio of the threshold distance with respect to one or more of:
   a length of the first-edge line;
   a diameter of a pupil in instances in which the transition edge corresponds to the pupil;
   a major axis of the pupil;
   a minor axis of the pupil;
   a diameter of a portion of the eye that excludes a sclera of the eye in instances in which the transition edge corresponds to the sclera;
   a major axis of the portion that excludes the sclera; or
   a minor axis of the portion that excludes the sclera;
an effect of an accuracy of the alignment registration by an amount of first-portion pixel data that is used to determine the first transition-edge representation, in which the amount of first-portion pixel data that is able to be used is based on a size of the first transition-edge portion; or
a user-selected distance.

Example 12. The system of any of Examples 9-11, wherein the identifying of the first-edge line based on the first-edge points includes using a plot of the first-edge points as the first-edge line.

Example 13. The system of any of Examples 9-11, wherein the identifying of the first-edge line based on the first-edge points includes:
applying a low-pass filter to a plot of the first-edge points to obtain a filtered plot of the first-edge points; and
using the filtered plot as the first-edge line.

Example 14. The system of any of Examples 9-13, wherein the identifying of the first transition-edge portion based on the first-edge line includes cropping out, based on the first-edge line, a particular portion of the first image.

Example 15. The system of Example 14, wherein the particular portion of the first image includes one or more of:
an inner-pupil portion of the first image that corresponds to an inner part of the pupil; or
an iris portion of the first image that corresponds to an iris of the eye.

Example 16. The system of any of Examples claim 1-15, wherein:
the operations further comprise determining a correlation between the first transition-edge representation and the second transition-edge representation based on the comparison; and
the alignment registration is determined based on the correlation.

Example 17. The system of Example 16, wherein the correlation includes a correlation distribution determined based on a plurality of correlations between a plurality of first-representation values corresponding to the first transition-edge representation and a plurality of second-representation values corresponding to the second transition-edge representation.

Example 18. The system of Example 17, wherein:
the plurality of first-representation values includes a plurality of first-edge intensity values determined from the first-portion pixel data; and
the plurality of second-representation values includes a plurality of second-edge intensity values determined from the second-portion pixel data.

Example 19. The system of any of Examples 17-18, wherein a subset of correlations of the plurality of correlations are individually and respectively determined based on a correlation determination between a subset of first-representation values and each respective second-representation value of the plurality of second-representation values.

Example 20. The system of any of Examples claim 16-19, wherein the correlation determination includes a cross-correlation determination.

Example 21. The system of any of Examples 1-20, wherein:
the first image of the eye corresponds to a first state of the eye; and
the second image of the eye corresponds to a second state of the eye.

Example 22. The system of Example 21, wherein:
the first state includes one or more of:
   a first dilation state;
   the eye being oriented in a first orientation;
   a first lighting condition associated with the eye;
   the eye being oriented according to a corresponding head being in an upright state; or
   the eye being oriented according to the corresponding head being in a laid-down state; and
the second state includes one or more of:
   a second dilation state;
   the eye being oriented in a second orientation;
   a second lighting condition associated with the eye;
   the eye being oriented according to the corresponding head being in the upright state; or
   the eye being oriented according to the corresponding head being in the laid-down state.

Example 23. The system of any of Examples 1-22, wherein:
the first transition-edge portion is a radial portion that corresponds to a first range of angular location values corresponding to a first polar coordinate system established with respect to the first image; and
the second transition-edge portion is a radial portion that corresponds to a second range of angular location values corresponding to a second polar coordinate system established with respect to the second image.

Example 24. The system of Example 23, wherein the first range of angular location values is the same as the second range of angular location values.

Example 25. The system of any of Examples 23-24, wherein the first range of angular location values corresponds to less than a full circumference of the transition edge.

Example 26. The system of any of Examples 23-25, wherein the first transition-edge portion is identified based on intensity value differences of intensity values respectively corresponding to a plurality of angular locations corresponding to the first image.

Example 27. The system of Example 26, wherein the first transition-edge portion is identified based on respective highest intensity value differences respectively corresponding to each different angular location and respectively corresponding to points of the first image that respectively correspond to a same angular location of the plurality of angular locations.

Example 28. The system of any of Examples 1, 2, and 4-27, wherein:
the transition edge corresponds to an outer edge of a pupil of the eye; and
the operations further comprise:
   identifying a first scleral-edge portion of the first image, the first scleral-edge portion corresponding to an inner edge of a sclera of the eye;
   determining, based on first sclera-portion pixel data corresponding to the first scleral-edge portion, a first scleral-edge representation of the inner edge of the sclera;
   identifying a second scleral-edge portion of the second image, the second scleral-edge portion corresponding to the inner edge of the sclera; and
   determining, based on second sclera-portion pixel data corresponding to the second scleral-edge portion, a second scleral-edge representation of the inner edge of the sclera; and
   the determining of the alignment registration is further based on a comparison between the first scleral-edge representation and the second scleral-edge representation.

Example 29. The system of Example 28, wherein using an iris of the eye is omitted from being used in determining the alignment registration.

Example 30. The system of any of Examples 1-29, wherein the determining of the alignment registration is further based on a point-based registration that is based on a first individual point corresponding to the eye in the first image and a second individual point corresponding to the eye in the second image.

Example 31. The system of Example 30, wherein the transition-edge based registration is weighted differently than the point-based registration in the determining of the alignment registration.

Example 32. The system of any of Examples 30-31, wherein the point-based registration is based on the first point being the brightest point in the first image corresponding to an iris of the eye and the second point being the brightest point in the second image corresponding to the iris.

Example 33. The system of any of Examples 30-31, wherein the point-based registration is based on the first point being the darkest point in the first image corresponding to an iris of the eye and the second point being the darkest point in the second image corresponding to the iris.

Example 34. The system of any of Examples 1-33, further comprising a laser configured to emit a laser beam used to perform one or more ophthalmic treatment operations, the laser beam being adjusted based on the alignment registration.

Example 35. The system of Example 34, wherein the adjustment of the laser beam includes adjusting an orientation of the laser beam with respect to the eye.

Example 36. The system of Example 35, wherein adjusting the orientation of the laser beam with respect to the eye includes one or more of:
adjusting a position of the laser beam with respect to the eye; or
adjusting a pattern of the laser beam.

Example 37. The system of Example 36, further comprising one or more actuators configured to adjust the position of the laser beam.

Example 38. The system of Example 37, wherein the one or more actuators are configured to adjust the position of the laser beam via one or more of:
adjusting one or more reflectors that reflect at least a portion of the laser beam;
adjusting an overall position of the laser; or
adjusting a position of a support on which a patient corresponding to the eye is laying.

Example 39. The system of any of Examples 34-38, further comprising a patient interface configured to interface with the eye and configured to have the laser beam pass therethrough.

Example 40. The system of any of Examples 1-39, wherein the first image is captured using a first camera separate from the system.

Example 41. The system of any of Examples 1-40, further comprising a second camera configured to capture the second image.

Example 42. A method performed by the system of any of claims 1-41.

What is claimed is:

1. A system comprising:
a computing system configured to cause performance of operations, the operations comprising:
identifying a first transition-edge portion of a first image of an eye, the first transition-edge portion corresponding to a transition edge of the eye;
determining, based on first-portion pixel data corresponding to the first transition-edge portion, a first transition-edge representation of the transition edge;
identifying a second transition-edge portion of a second image of the eye, the second transition-edge portion corresponding to the transition edge of the eye;
determining, based on second-portion pixel data corresponding to the second transition-edge portion, a second transition-edge representation of the transition edge; and
determining an alignment registration between the eye as depicted in the first image and the eye as depicted in the second image based on a transition-edge based registration that is based on a comparison between the first transition-edge representation and the second transition-edge representation.

2. The system of claim 1, wherein the transition edge corresponds to an outer edge of a pupil of the eye.

3. The system of claim 1, wherein the transition edge corresponds to an inner edge of a sclera of the eye.

4. The system of claim 1, wherein:
the determining of the first transition-edge representation based on the first-portion pixel data is based on a plurality of first-edge intensity values determined from the first-portion pixel data; and
the determining of the second transition-edge representation based on the second-portion pixel data is based on a plurality of second-edge intensity values determined from the second-portion pixel data.

5. The system of claim 4, wherein the first-edge intensity values include a plurality of aggregate first-edge intensity values that individually correspond to a respective first sub-portion of a plurality of first sub-portions of the first transition-edge portion, each aggregate first-edge intensity value being based on first sub-portion intensity values of its corresponding first sub-portion; and
determining the first transition-edge representation based on the plurality of aggregate first-edge intensity values.

6. The system of claim 5, wherein each first sub-portion of the plurality of first sub-portions includes a slice of pixels extending radially outward from an inner edge of the first transition-edge portion to an outer edge of the first transition-edge portion.

7. The system of claim 5, wherein one or more of the first-edge intensity values are based on one or more of:
an average of the first sub-portion intensity values of their corresponding first sub-portions;
a sum of the first sub-portion intensity values of their corresponding first sub-portions; or
one or more weight values that are respectively applied to one or more of the first sub-portion intensity values.

8. The system of claim 7, wherein the one or more weight values are based on a distance away from an identified center of a pupil of the eye.

9. The system of claim 1, wherein the identifying of the first transition-edge portion includes:
identifying first-edge points in the first image, the first-edge points corresponding to the transition edge;
identifying, in the first image, a first-edge line corresponding to the transition edge based on the first-edge points; and
identifying the first transition-edge portion based on the first-edge line.

10. The system of claim 9, wherein the identifying of the first transition-edge portion based on the first-edge line includes identifying the first transition-edge portion based on a threshold distance radially extended away from the first-edge line.

11. The system of claim 10, wherein the threshold distance is based on one or more of:
a number of pixels in the first image away from the first-edge line;
a ratio of the threshold distance with respect to one or more of:
a length of the first-edge line;
a diameter of a pupil in instances in which the transition edge corresponds to the pupil;
a major axis of the pupil;
a minor axis of the pupil;
a diameter of a portion of the eye that excludes a sclera of the eye in instances in which the transition edge corresponds to the sclera;
a major axis of the portion that excludes the sclera; or
a minor axis of the portion that excludes the sclera;
an effect of an accuracy of the alignment registration by an amount of first-portion pixel data that is used to determine the first transition-edge representation, in which the amount of first-portion pixel data that is able to be used is based on a size of the first transition-edge portion; or
a user-selected distance.

12. The system of claim 9, wherein the identifying of the first-edge line based on the first-edge points includes using a plot of the first-edge points as the first-edge line.

13. The system of claim 9, wherein the identifying of the first-edge line based on the first-edge points includes:
applying a low-pass filter to a plot of the first-edge points to obtain a filtered plot of the first-edge points; and
using the filtered plot as the first-edge line.

14. The system of claim 9, wherein the identifying of the first transition-edge portion based on the first-edge line includes cropping out, based on the first-edge line, a particular portion of the first image.

15. The system of claim 14, wherein the particular portion of the first image includes one or more of:
an inner-pupil portion of the first image that corresponds to an inner part of the pupil; or
an iris portion of the first image that corresponds to an iris of the eye.

16. The system of claim 1, wherein:
the operations further comprise determining a correlation between the first transition-edge representation and the second transition-edge representation based on the comparison; and
the alignment registration is determined based on the correlation.

17. The system of claim 16, wherein the correlation includes a correlation distribution determined based on a plurality of correlations between a plurality of first-representation values corresponding to the first transition-edge representation and a plurality of second-representation values corresponding to the second transition-edge representation.

18. The system of claim 17, wherein:
the plurality of first-representation values includes a plurality of first-edge intensity values determined from the first-portion pixel data; and
the plurality of second-representation values includes a plurality of second-edge intensity values determined from the second-portion pixel data.

19. The system of claim 17, wherein a subset of correlations of the plurality of correlations are individually and respectively determined based on a correlation determination between a subset of first-representation values and each respective second-representation value of the plurality of second-representation values.

20. The system of claim 16, wherein the correlation determination includes a cross-correlation determination.

21. The system of claim 1, wherein:
the first image of the eye corresponds to a first state of the eye; and
the second image of the eye corresponds to a second state of the eye.

22. The system of claim 21, wherein:
the first state includes one or more of:
a first dilation state;
the eye being oriented in a first orientation;
a first lighting condition associated with the eye;
the eye being oriented according to a corresponding head being in an upright state; or
the eye being oriented according to the corresponding head being in a laid-down state; and
the second state includes one or more of:
a second dilation state;
the eye being oriented in a second orientation;
a second lighting condition associated with the eye;
the eye being oriented according to the corresponding head being in the upright state; or
the eye being oriented according to the corresponding head being in the laid-down state.

23. The system of claim 1, wherein:
the first transition-edge portion is a radial portion that corresponds to a first range of angular location values corresponding to a first polar coordinate system established with respect to the first image; and
the second transition-edge portion is a radial portion that corresponds to a second range of angular location values corresponding to a second polar coordinate system established with respect to the second image.

24. The system of claim 23, wherein the first range of angular location values is the same as the second range of angular location values.

25. The system of claim 23, wherein the first range of angular location values corresponds to less than a full circumference of the transition edge.

26. The system of claim 23, wherein the first transition-edge portion is identified based on intensity value differences of intensity values respectively corresponding to a plurality of angular locations corresponding to the first image.

27. The system of claim 26, wherein the first transition-edge portion is identified based on respective highest intensity value differences respectively corresponding to each different angular location and respectively corresponding to points of the first image that respectively correspond to a same angular location of the plurality of angular locations.

28. The system of claim 1, wherein:
the transition edge corresponds to an outer edge of a pupil of the eye; and
the operations further comprise:
identifying a first scleral-edge portion of the first image, the first scleral-edge portion corresponding to an inner edge of a sclera of the eye;
determining, based on first sclera-portion pixel data corresponding to the first scleral-edge portion, a first scleral-edge representation of the inner edge of the sclera;
identifying a second scleral-edge portion of the second image, the second scleral-edge portion corresponding to the inner edge of the sclera; and
determining, based on second sclera-portion pixel data corresponding to the second scleral-edge portion, a second scleral-edge representation of the inner edge of the sclera; and
the determining of the alignment registration is further based on a comparison between the first scleral-edge representation and the second scleral-edge representation.

29. The system of claim 28, wherein using an iris of the eye is omitted from being used in determining the alignment registration.

30. The system of claim 1, wherein the determining of the alignment registration is further based on a point-based registration that is based on a first individual point corresponding to the eye in the first image and a second individual point corresponding to the eye in the second image.

31. The system of claim 30, wherein the transition-edge based registration is weighted differently than the point-based registration in the determining of the alignment registration.

32. The system of claim 30, wherein the point-based registration is based on the first individual point being the brightest point in the first image corresponding to an iris of the eye and the second individual point being the brightest point in the second image corresponding to the iris.

33. The system of claim 30, wherein the point-based registration is based on the first individual point being the darkest point in the first image corresponding to an iris of the eye and the second individual point being the darkest point in the second image corresponding to the iris.

34. The system of claim 1, further comprising a laser configured to emit a laser beam used to perform one or more ophthalmic treatment operations, the laser beam being adjusted based on the alignment registration.

35. The system of claim 34, wherein the adjustment of the laser beam includes adjusting an orientation of the laser beam with respect to the eye.

36. The system of claim 35, wherein adjusting the orientation of the laser beam with respect to the eye includes one or more of:
adjusting a position of the laser beam with respect to the eye; or
adjusting a pattern of the laser beam.

37. The system of claim 36, further comprising one or more actuators configured to adjust the position of the laser beam.

38. The system of claim 37, wherein the one or more actuators are configured to adjust the position of the laser beam via one or more of:
adjusting one or more reflectors that reflect at least a portion of the laser beam;
adjusting an overall position of the laser; or
adjusting a position of a support on which a patient corresponding to the eye is laying.

39. The system of claim 34, further comprising a patient interface configured to interface with the eye and configured to have the laser beam pass therethrough.

40. The system of claim 1, wherein the first image is captured using a first camera separate from the system.

41. The system of claim 40, further comprising a second camera configured to capture the second image.
